SEQUENCE LISTING (50 sequences)

SEQ ID NO: 1 - protein, 2351 aa, Homo sapiens [sequence omitted]

SEQ ID NO: 2 - DNA, 7056 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 3 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - DNA, 7056 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 5 - protein, 2350 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPSNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKKVFQGNQDSFTPVVNSLDPPLLTRYLRIHPQSWVHQIALRMEVLGCEAQDLY

SEQ ID NO: 6 - protein, 14 aa, Homo sapiens
SFSQNPPVLKRHQR

SEQ ID NO: 7 - protein, 1438 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - DNA, 696 bp, Homo sapiens
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 9 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 684 bp, Homo sapiens
gagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 11 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - DNA, 837 bp, Homo sapiens
gagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 13 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - DNA, 687 bp, Homo sapiens
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 651 bp, Homo sapiens
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 648 bp, Homo sapiens
gcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 19 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - DNA, 627 bp, Homo sapiens
tcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 21 - protein, 209 aa, Homo sapiens
SVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - DNA, 651 bp, Homo sapiens
gcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 23 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 11 aa, Artificial Sequence
  FcRn binding peptide
PKNSSMISNTP

SEQ ID NO: 25 - protein, 7 aa, Artificial Sequence
  FcRn binding peptide
HQSLGTQ

SEQ ID NO: 26 - protein, 8 aa, Artificial Sequence
  FcRn binding peptide
HQNLSDGK

SEQ ID NO: 27 - protein, 8 aa, Artificial Sequence
  FcRn binding peptide
HQNISDGK

SEQ ID NO: 28 - protein, 8 aa, Artificial Sequence
  FcRn binding peptide
VISSHLGQ

SEQ ID NO: 29 - protein, 227 aa, Artificial Sequence
  Mouse Fc Region of IgG1
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 30 - protein, 214 aa, Artificial Sequence
  Mouse Fc-Hinge region of IgG1
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 31 - DNA, 5061 bp, Artificial Sequence
  BDDmFc + hinge
atggaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctcccagaacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatcccagaacccaccagtccttaagcgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 32 - protein, 1687 aa, Artificial Sequence
  BDDmFC+hinge
MEIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 33 - protein, 1674 aa, Artificial Sequence
  BDDmFC-hinge
MEIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 34 - DNA, 62 bp, Artificial Sequence
  Mutagenic primer CES16
caatgccattgaacctaggagcttctcccagaacccaccagtccttaagcgccatcaacggg

SEQ ID NO: 35 - DNA, 62 bp, Artificial Sequence
  Mutagenic primer CES17
cccgttgatggcgcttaaggactggtgggttctgggagaagctcctaggttcaatggcattg

SEQ ID NO: 36 - DNA, 37 bp, Artificial Sequence
  Mutagenic oligonucleotide CES18
cagtggtcattacactcaagaacatggcttcccatcc

SEQ ID NO: 37 - DNA, 37 bp, Artificial Sequence
  Mutagenic oligonucleotide CES19
ggatgggaagccatgttcttgagtgtaatgaccactg

SEQ ID NO: 38 - DNA, 46 bp, Artificial Sequence
  Primer CES36
agcttcctaggagcttctcccagaacgtgcccagggattgtggttg

SEQ ID NO: 39 - DNA, 50 bp, Artificial Sequence
  Primer CES39
agctacttaaggactggtgggttctgggatttaccaggagagtgggagag

SEQ ID NO: 40 - DNA, 47 bp, Artificial Sequence
  Primer CES37
agcttcctaggagcttctcccagaacgtcccagaagtatcatctgtc

SEQ ID NO: 41 - DNA, 129 bp, Artificial Sequence
  Primer CES49
atatgatatcgcggccgccgccaccatggtgttgcagacccaggtcttcatttctctgttgctctggatctctggtgcctacggggactacaaagacgatgacgacaaggtgcccagggattgtggttg

SEQ ID NO: 42 - DNA, 37 bp, Artificial Sequence
  Primer CES50
ttcgatctcgagtcatttaccaggagagtgggagagg

SEQ ID NO: 43 - DNA, 36 bp, Artificial Sequence
  Primer CES51
cagttgccgcgggctttaccaggagagtgggagagg

SEQ ID NO: 44 - DNA, 62 bp, Artificial Sequence
  Primer CES52
ttcgcccgcggcaagagagactacaaagacgatgacgacaaggtgcccagggattgtggttg

SEQ ID NO: 45 - protein, 6 aa, Artificial Sequence
  Furin consensus sequence
KARGKR

SEQ ID NO: 46 - protein, 8 aa, Artificial Sequence
  Flag Tag sequence
DYKDDDDK

SEQ ID NO: 47 - DNA, 681 bp, Mouse
gtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 48 - DNA, 642 bp, Mouse
gtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 49 - protein, 5 aa, Artificial Sequence
  Peptide linker
DDDDK

SEQ ID NO: 50 - protein, 15 aa, Artificial Sequence
  Peptide Linker
GGGGSGGGGSGGGGS

(19) United States
(12) Patent Application Publication
(10) Pub. No.: US 2012/0142593 A1
(43) Pub. Date: Jun. 7, 2012

(54) Factor VIII Variants and Methods of Use

(75) Inventors: Xiao-Yan Zhao, Union City, CA (US); Peter John Kretschmer, San Francisco, CA (US); Thomas Eugene Thompson, Alameda, CA (US); Douglas W. Schneider, Lafayette, CA (US); John Edward Murphy, Berkeley, CA (US)

(73) Assignee: BAYER HEALTHCARE LLC, Tarrytown, NY (US)

(21) Appl. No.: 13/260,564

(22) PCT Filed: Mar. 24, 2010
(86) PCT No.: PCT/US10/28529

§ 371 Date: Jan. 4, 2012

Related U.S. Application Data
(60) Provisional application No. 61/162,986, filed on Mar. 24, 2009.

(51) Int. Cl. A61K 38/37; C12N 15/62; C12N 15/63; C12N 5/10; A61P 7/04; C12N 1/15; C12N 1/19; C12N 1/13; C12P 21/00; C07K 19/00; C12N 1/21

(52) U.S. Cl. 514/14.1; 530/383; 536/23.4; 435/320.1; 435/325; 435/252.3; 435/252.33; 435/348; 435/254.11; 435/254.2; 435/254.21; 435/419; 435/414; 435/257.2; 435/352; 435/358; 435/367; 435/369; 435/355; 435/346; 435/69.6

(57) ABSTRACT

A Factor VIII fusion protein or a Factor VIII fusion heterodimer comprising Factor VIII in which an amino acid sequence of a modulator is present in the B-domain, or an amino acid sequence of a modulator replaces some or all of the amino acid sequence of the B-domain is disclosed. Nucleic acids encoding the inventive fusion proteins and fusion heterodimers are also disclosed, as are methods for producing the fusion proteins and fusion heterodimers, pharmaceutical compositions, and methods of treating deficiencies in coagulation with the inventive fusion molecules.

This application claims benefit of U.S. Provisional Application Ser. No. 61/162,986; filed on Mar. 24, 2009, the contents of which are incorporated herein by reference in their entirety.

### FIELD OF THE INVENTION

This invention relates to variant Factor VIII (FVIII) proteins. This invention also relates to nucleic acids coding for variant FVIII proteins and methods for identifying such nucleic acids. The present invention relates to methods of making and using the variant FVIII proteins.

### BACKGROUND OF THE INVENTION

Coagulation of blood occurs by either the contact activation pathway (formerly known as the intrinsic pathway) or the tissue factor pathway (formerly known as the extrinsic pathway), whereby certain blood proteins interact in a cascade of proteolytic activations to ultimately convert soluble fibrinogen to insoluble fibrin. These threads of fibrin are cross-linked to form the scaffolding of a clot; without fibrin formation, coagulation cannot occur.

The contact activation pathway consists of several steps: (1) the proteolytic activation of Factor XII; (2) activated Factor XII cleaves Factor XI to activate it; (3) activated Factor XI cleaves Factor IX, thereby activating it; (4) activated Factor IX interacts with activated FVIII to cleave and activate Factor X; (5) activated Factor X binds to activated Factor V on a membrane surface, which complex proteolytically cleaves prothrombin to form thrombin; (6) thrombin proteolytically cleaves fibrinogen to form fibrin; (7) fibrin monomers assemble into fibrils, which are then cross-linked by Factor XIII.

The tissue factor pathway consists of the following steps: (1) upon rupture of a blood vessel, Factor VII binds to tissue factor, a lipoprotein present in tissues outside the vascular system; (2) Factor VII is activated to Factor VIIa by proteolytic cleavage; and (3) the Factor VIIa-tissue factor complex cleaves and activates Factor X. Thereafter, the tissue factor pathway is identical to the contact activation pathway, that is, the two pathways share the last three steps described above.

The biosynthesis, intracellular processing, and secretion of FVIII, and the mechanism by which it subsequently becomes activated in blood plasma is well known in the art (see, e.g., Lenting, et al., Blood 92:3983-3996, 1998; Thompson, Seminars in Hemostasis 29:11-22, 2003; Graw, et al., Nature Reviews: Genetics 6:489-501, 2005). Human FVIII is initially translated as a single chain polypeptide of 2351 amino acids (SEQ ID NO: 1), with the first 19 amino acids defining a signal peptide that is removed by a signal peptidase within the ER. Mature human FVIII thus consists of 2332 amino acids with domain structure A1-a1-A2-a2-B-a3-A3-C1-C2 (FIG. 1A). FVIII is glycosylated and processed intracellularly prior to secretion by cleavage near the carboxy-terminus of the B domain (Arg-1648, at the B-a3 junction), and is variably cleaved within the B domain, predominantly after Arg-1313, to produce a 90-210 kDa heavy chain and an 80 kDa light chain (FIG. 1B). FVIII is thereafter secreted as a heterodimer glycoprotein consisting of a single heavy chain and single light chain.

The plasma glycoprotein FVIII circulates as an inactive precursor in blood, bound tightly and non-covalently to von Willebrand factor (vWf). FVIII is proteolytically activated by cleavage by thrombin or Factor Xa at three Arg-Ser peptide bonds, namely after Arg-372, Arg-740, and Arg 1689, which dissociates it from vWf and activates its procoagulant function in the cascade. The resulting heterotrimer becomes FVIIIa (FIG. 1C).

In its active form (i.e., FVIIIa), FVIII functions as a cofactor for the Factor X activation enzyme complex in the contact activation pathway of blood coagulation, and it is decreased or nonfunctional in patients with hemophilia A. The level of the decrease in FVIII activity is directly proportional to the severity of the disease. Thus, people with deficiencies in FVIII or with antibodies against FVIII suffer uncontrolled internal bleeding that may cause a range of serious symptoms unless they are treated with FVIII. Symptoms range from inflammatory reactions in joints to early death. The classic definition of FVIII, in fact, is that substance present in normal blood plasma that corrects the clotting defect in plasma derived from individuals with hemophilia A. A deficiency in vWf can also cause phenotypic hemophilia A because vWf is an essential component of functional FVIII. In these cases, the circulating half-life of FVIII in plasma is decreased to such an extent that it can no longer perform its particular functions in blood clotting. The current treatment of hemophilia A consists of the replacement of the missing protein by administration of plasma-derived or recombinant FVIII.

The development of antibodies (“inhibitors” or “inhibitory antibodies”) that inhibit the activity of FVIII is a serious complication in the management of patients with hemophilia A. Autoantibodies develop in approximately 20% of patients with hemophilia A in response to therapeutic infusions of FVIII. In previously untreated patients with hemophilia A who develop inhibitors, the inhibitor usually develops within one year of treatment. Additionally, autoantibodies that inactivate FVIII occasionally develop in individuals with previously normal FVIII levels. If the inhibitor titer is low enough, patients can be managed by increasing the dose of FVIII. However, often the inhibitor titer is so high that it cannot be overwhelmed by FVIII. An alternative strategy is to bypass the need for FVIII during normal hemostasis using Factor IX complex preparations or recombinant human Factor VIIa. Additionally, since porcine FVIII usually has substantially less reactivity with inhibitors than human FVIII, a partially purified porcine FVIII preparation may be used. Many patients who have developed inhibitory antibodies to human FVIII have been successfully treated with porcine FVIII and have tolerated such treatment for long periods of time. However, administration of porcine FVIII is not a complete solution because inhibitors may develop to porcine FVIII after one or more infusions. Thus, the use of recombinant human FVIII or partially-purified porcine FVIII has not resolved all the problems.

In addition to inhibitory antibodies, problems also arise in that FVIII, when administered intravenously, has a relatively short half-life in circulation (13 hours in human), so frequent infusions are needed, which causes difficulty in patient dosing compliance. A longer acting FVIII for weekly dosing or even monthly dosing is thus an unmet medical need (Dargaud, et al., Expert Opinion on Biological Therapy 7:651-663, 2007). Longer protection would be achieved by prolonging FVIII half-life. A number of FVIII bioengineering approaches are being explored with the goal of producing protection for longer periods of time (Baru, et al., Thromb. Haemost. 93:1061-1068, 2005; Pipe, J. Thromb. Haemost. 3:1692-1701, 2005; Saenko, et al., Haemophilia 12(Suppl 3):42-51, 2006).

The present invention relates FVIII variants which demonstrate modified activity and/or modified pharmacokinetic properties (e.g., longer circulating half-life). As an example, the FVIII variant may be a fusion or heterodimer protein where an amino acid sequence (e.g., modulator) is either inserted in the B-domain portion of the FVIII protein or the B-domain or a portion of the B-domain is replaced with this amino acid sequence. This insertion/replacement amino acid sequence does not disrupt the post-translational processing of FVIII and this FVIII variant has activity as a coagulation factor. These FVIII variants may be used to treat hemophilia A, and may lead to less frequent administration due to, for example, a longer circulating half-life. By requiring less frequent dosing, the FVIII variants of the invention may improve patient compliance and reduce the likelihood of a patient developing an immune response to the FVIII because FVIII is administered.

### SUMMARY OF THE INVENTION

The present invention relates to FVIII fusion proteins and expression products thereof (also referred to herein as FVIII fusion heterodimers). The present invention further relates to hybrid FVIII fusion heterodimers and multimeric FVIII fusion heterodimers. In one embodiment, the FVIII fusion heterodimer comprises a FVIII protein or polypeptide and an amino acid sequence (referred to herein as modulator). In another embodiment, the modulator sequence is inserted into the FVIII B domain. In further embodiment, at least a portion of the B domain is deleted and replaced by the modulator sequence.

The present invention also relates to the nucleic acid sequences encoding the FVIII fusion heterodimers. In one embodiment, the nucleic acid sequence encodes a FVIII fusion heterodimer comprising a FVIII protein in which a modulator sequence is present in the B domain or a modulator sequence replaces some or all of the amino acid sequence of the B domain. The nucleic acid sequence encoding the FVIII fusion heterodimers may be operatively linked in an expression cassette. The present invention also includes methods of making FVIII fusion heterodimers. For example, an expression cassette encoding a FVIII fusion heterodimer, if not already a part of an expression vector, is introduced into an expression vector and subsequently introduced into an appropriate host cell for recombinant production of the FVIII fusion heterodimers. The fusion heterodimers produced have FVIII activity in vitro and in vivo and may, for example, display increased circulating half-life in vivo.

In a further embodiment of the present invention, a FVIII fusion heterodimer comprises a first amino acid sequence corresponding to amino acids 20-764 of any one of SEQ ID NO: 1, 3, or 5; a second amino acid sequence corresponding to amino acids 1656-2351 of any one of SEQ ID NO: 1, 3, or 5; and a modulator sequence in which (1) the modulator sequence is covalently attached at its amino terminal to the carboxyl terminal of the first amino acid sequence and covalently attached at its carboxyl terminal to the amino terminal of the second amino acid, or (2) the modulator sequence is covalently attached at its amino terminal to the carboxyl terminal of the first amino acid sequence and the modulator sequence is not covalently attached to the second amino acid sequence.

In another embodiment of the present invention, a nucleic acid sequence encodes a FVIII fusion heterodimer, wherein the FVIII fusion heterodimer comprises a first amino acid sequence corresponding to amino acids 20-764 of any one of SEQ ID NO: 1, 3, or 5; a second amino acid sequence corresponding to amino acids 1656-2351 of any one of SEQ ID NO: 1, 3, or 5; and a modulator sequence in which the modulator sequence is covalently attached at its amino terminal to the carboxyl terminal of the first amino acid sequence and covalently attached at its carboxyl terminal to the amino terminal of the second amino acid. In addition, the present invention also relates to vectors, host cells, methods of producing fusion heterodimers and methods of treating coagulation deficiencies.

### DESCRIPTION OF THE DRAWINGS

FIG. 1A illustrates the structure of full-length human FVIII which contains from N-terminal to C-terminal the following domains: S (signal peptide), A1, a1, A2, a2, B, a3, A3, C1, and C2. FIG. 1B illustrates the structure of the heavy and light chains of heterodimeric human Factor VIII. The size of the heavy chain varies as a result of variable proteolytic cleavage within the B-domain. FIG. 1C illustrates the structure of the subunits of active human FVIII (i.e., FVIIIa).

FIG. 2 illustrates three exemplary embodiments of Factor VIII fusion heterodimers of the present invention described in the examples section. The three embodiments are denoted “BDDFc+hinge,” “BDDFc−hinge,” and “BDDFc” (which may optionally comprise a heterologous peptide tag to facilitate isolation). The three exemplary embodiments differ in their ability to form dimers (via their Fc portion) or protein aggregates and in their binding affinity for FcRn.

FIG. 3 describes the structural domains of the Factor VIII fusion proteins produced in accordance with Examples 1 and 2. Specifically, a murine Fc region (with or without a hinge) was inserted into the specific site (between N-745 and S-1637) of a B-domain deleted (BDD) Factor VIII protein to replace the deleted portion of the B-domain. The amino acid sequences of the non-deleted B-domain portions on the N-terminal and C-terminal sides of the murine Fc region are indicated.

FIG. 4 illustrates the monocistronic BDD.mFc monomer construct produced in accordance with Example 5.

FIG. 5 illustrates identification of high-expression clones by activity assays. HKB11 stable cell lines expressing BDDFc+hinge were screened by FVIII aPPT coagulation assays. Clones (4, 8, 12, 18, 27, and 33) showed high coagulation activities ranging from 500-3500 mIU/mL.

FIG. 6 illustrates identification of high-expression clones by ELISA assays. HKB11 stable cell lines expressing BDDFc+hinge were screened by anti-FVIII capture ELISA. Three clones (clone 8, 18, and 27) express at ˜1 ug/mL BDDFc+hinge fusion.

FIG. 7 shows the results of protein purification of BDDFc+hinge fusion proteins. In the reduced gel, BDDFc+hinge was resolved as an 80-kDa Light chain (L), a 115-kDa heavy chain (H), and a 195-kDa unprocessed single chain (U) (lane 8). In the non-reduced gel, BDDFc+hinge produced a 390-kDa band (dimer) in addition to the 80-, 115-, 195-kDa bands (lane 8).

FIG. 8 demonstrates the recovery of BDDFc−hinge (“FVIII-Fc”) and BDD-FVIII in hemophilia A (Hem A) mice. Nine HemA mice received 50 μg/kg (400 IU/kg) () of BDDFc-hinge in formulation buffer containing 5% albumin. Additional HemA mice received 200 IU/kg (□) of BDD-FVIII, the Factor VIII variant from which BDDFc−hinge is derived. In comparison to the decay curve of BDD-FVIII, BDDFc−hinge showed biphasic decay with a rapid distribution phase. The beta phase half-life of BDDFc−hinge was 11.9 hrs at 50 μg/kg, which is about a 2-fold improvement relative to unmodified BDD-FVIII for which the beta phase half-life is 6.03 hrs.

FIG. 9A illustrates the BDD-Fc chimeric chain of BDDFc+hinge detected as a 115 kDa band in Western blot analyses. Samples from both transient transfectants (trans) and stable pools (sp) were concentrated 5-fold then run on 10% NuPAGE® gels under reducing conditions. Lanes: 1) molecular weight markers; 2) purified BDD protein as standard; 3-5) concentrated conditioned media from HKB11 cells transiently transfected with pSK207 vector, pSK207BDD, and pSK207BDDFc+hinge, respectively; 7-9) concentrated conditioned media from stable pools of HKB11 cells stably transfected with pSK207 vector, pSK207BDD, pSK207BDDFc+hinge, respectively. The blot was probed with HRP-conjugated anti-mouse IgG (H+ L). An unprocessed single-chain form of BDDFc+hinge (“sc BDD-Fc”) was detected as a 195 kDa band, and the heterodimeric form of BDDFc+hinge comprises a 115 kDa chimera of Factor VIII heavy chain and Fc (“Heavy chain Fc”). No band appears for the light chain of heterodimeric BDDFc+hinge since it is not bound by HRP-conjugated anti-mouse IgG. FIG. 9B shows a BDDFc light chain detected as a 80 kDa band in Western blot analyses. Protein samples were run on 10% NuPAGE® gels under reducing conditions. Lanes: 1) molecular weight markers; 2) purified BDD protein as standard; 3-5) concentrated conditioned media from HKB11 cells transiently transfected with pSK207 vector, pSK207BDD, and pSK207BDDFc+hinge, respectively. The blot was probed with FVIII light chain specific antibody.

FIG. 10 shows the results of Factor VIII activity assays. Conditioned media from HKB11 cells transiently transfected with pSK207BDDFc+hinge (“Fc+Hinge sup”) and pSK207BDDFc−hinge (“Fc−Hinge sup”) were collected and tested for FVIII activity in both Coatest® assay and in aPPT coagulation assays. As controls, vectors pSK207 and pSK207BDD (“BDD sup”) which encodes the unmodified Factor VIII protein, were used in transfections as well as in activity assays.

FIG. 11 shows Factor VIII activity for the Factor VIII fusion heterodimers. Conditioned media from HKB11 cells [(BDDFc+hinge transient transfectants (Tr) and stable pools (Sp)] were loaded onto a 96-well plate pre-coated with rabbit-anti-mouse Fc antibody. After a 2-hour incubation at room temperature, the plate was washed three times with PBS/Tween®-20/BSA to remove non-specific binding prior to Coatest® assays.

FIG. 12 demonstrates that BDDFc+hinge form dimmers and BDDFc−hinge is a monomer. Western blot analyses were performed using 5-fold concentrated conditioned media from HKB11 cells transfected with pSK207BDDFc+hinge or pSK207BDDFc−hinge expression vector. Samples were run on 4-12% NuPAGE® gels under reducing and non-reducing conditions. The blot was probed with rabbit monoclonal anti-FVIII light chain antibody (Epitomics, Burlingame, Calif.) followed by HRP-conjugated anti-rabbit IgG secondary antibody. The unprocessed single-chain BDD and BDDFc were detected as 170-kDa and 195-kDa bands, respectively. Lanes: BDD—purified BDD protein; +H—BDDFc+hinge; —H—BDDFc−hinge; and V—pSK207 vector alone.

FIG. 13 shows the results of a Biacore™ study measuring the ability of BDDFc+hinge and BDDFc−hinge (“BDDFc−H”) which incorporate a mouse FcRn binding epitope, to bind to immobilized mouse FcRn. BDDFc+hinge (“BDDFc+H”), BDDFc−hinge (“BDDFc−H”), BDD, and full-length recombinant Factor VIII (“FVIII”). No detectable binding was seen with BDD or full length Factor VIII. BDDFc+hinge and BDDFc−hinge fusion proteins showed strong binding for mFcRn with nM affinity.

FIG. 14 shows the results of a Biacore™ study measuring the ability of BDDFc+hinge and BDDFc−hinge to bind to immobilized human von Willebrand Factor (vWF). Mouse FcRn was immobilized onto a CM-5 chip by amine coupling. BDDFc+hinge (“BDDFc+H”), BDDFc−hinge (“BDDFc−H”), BDD, and full-length recombinant Factor VIII (“FVIII”) show sub-nanomolar affinity for vWF.

FIG. 15 shows that BDDFc−hinge was efficacious in the tail vein transection bleeding model of HemA mice. To determine whether BDDFc−hinge is functional in treating bleeds in vivo, HemA mice were injected via the tail vein with BDDFc−hinge, BDD-FVIII, or vehicle control at 48 hrs prior to the transection of one lateral tail vein. In comparison to the vehicle-control group (▴) in which only 10% survived for 24 hrs following the injury, 12 IU/kg () and 60 IU/kg (▪) of BDDFc−hinge achieved 25% and 80% of survival, respectively. The efficacy of FVIII-Fc−hinge is estimated to be comparable to that of BDD-FVIII, which resulted in 60% survival at 40 IU/kg (♦).

### DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a protein” is a reference to one or more proteins and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

As used herein, various terms are defined below.

A “nucleic acid” denotes deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Degenerate codon substitutions may be achieved by generating sequences in which the third position of one or more selected (or all) codons is substituted with mixed-base and/or deoxyinosine residues. The term nucleic acid, depending on context, is used interchangeably with gene, cDNA, and mRNA encoded by a gene.

“Nucleic acid derived from a gene” denotes a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, and the like, are derived from the gene and detection of such derived products is indicative of the presence and/or abundance of the original gene and/or gene transcript in a sample.

A nucleic acid sequence is “operatively linked” or “operatively inserted” when it is placed into a functional relationship with another nucleic acid sequence. For example, a promoter or enhancer may be operatively linked to a coding sequence. Operatively linked nucleic acid sequences may be contiguous an/or join two protein coding regions. Some nucleic acid sequences may be operatively linked but not contiguous. Linking of nucleic acid sequences may be accomplished by ligation at restriction sites. If such sites do not exist, synthetic oligonucleotide adaptors or linkers may be used in accordance with conventional practice.

A first polypeptide having biological activity is “operatively linked” to a second polypeptide having biological activity when it is placed into a functional relationship with the second polypeptide such that at least a minimal level of the biological activity is retained by both the first polypeptide and the second polypeptide. In the context of polypeptides, operative linkage does not necessarily imply that the first and second polypeptide are contiguous. As one of skill in the art appreciates, maintenance of biological activities may be facilitated by inclusion of a peptide linker.

A polypeptide, nucleic acid, or other component is “isolated” when it is partially or completely separated from components with which it is normally associated (other peptides, polypeptides, proteins (including complexes, for example, polymerases and ribosomes which may accompany a native sequence), nucleic acids, cells, synthetic reagents, cellular contaminants, cellular components, etc.), for example, such as from other components with which it is normally associated in the cell from which it was originally derived. A polypeptide, nucleic acid, or other component is isolated when it is partially or completely recovered or separated from other components of its natural environment such that it is the predominant species present in a composition, mixture, or collection of components (i.e., on a molar basis it is more abundant than any other individual species in the composition). In some instances, the preparation consists of more than about 60%, 70% or 75%, typically more than about 80%, or more than about 90% of the isolated species.

A “substantially pure” nucleic acid (e.g., RNA or DNA), polypeptide, protein, or composition also means where the object species (e.g., nucleic acid or polypeptide) comprises at least about 50, 60, 70, 80, 90, or 95 percent by weight of all the macromolecular species present in the composition. An object species can also be purified to essential homogeneity (contaminant species cannot be detected in the composition by conventional detection methods) wherein the composition consists essentially of derivatives of a single macromolecular species.

The term “purified” generally means that the nucleic acid, polypeptide, or protein is at least about 50% pure, 60% pure, 70% pure, 75% pure, 85% pure, and 99% pure.

The term “recombinant” when used with reference, for example, to a cell, polynucleotide, vector, protein, or polypeptide typically denotes that the cell, polynucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences that may not be found in the native (non-recombinant) form of the cell or express native nucleic acid sequences that would otherwise be abnormally expressed, under-expressed, or not expressed at all. The term “recombinant” when used with reference to a cell indicates that the cell replicates a heterologous nucleic acid, or expresses a polypeptide encoded by a heterologous nucleic acid. Recombinant cells may contain coding sequences that are not found within the native (non-recombinant) form of the cell. Recombinant cells may also contain coding sequences found in the native form of the cell wherein the coding sequences are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, recombination, and related techniques.

The term “recombinantly produced” denotes an artificial combination usually accomplished by either chemical synthesis means, recursive sequence recombination of nucleic acid segments or other diversity generation methods (such as, e.g., shuffling) of nucleotides, or manipulation of isolated segments of nucleic acids, for example, by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated.

A “recombinant expression cassette” or simply an “expression cassette” denotes a nucleic acid construct, generated recombinantly or synthetically, with nucleic acid elements that are capable of effecting expression of a nucleic acid coding for a structural protein in hosts compatible with such sequences. An expression cassette necessarily includes a nucleic acid to be transcribed (e.g., a nucleic acid encoding a desired polypeptide), and a promoter. Additional components necessary or helpful in effecting expression may also be used as described herein. For example, an expression cassette may also include nucleotide sequences that encode a sorting signal (e.g., a signal peptide or secretory leader sequence) that directs secretion of an expressed protein from the host cell. Transcription termination signals, enhancers, and other nucleic acid sequences that influence gene expression, may also be included in an expression cassette. For purposes of the present invention, an “expression cassette comprising a Factor VIII fusion gene” indicates that the desired protein expressed by the expression cassette is a “Factor VIII fusion protein” as that term is defined further below.

The term “vector” may refer to, depending on context, cloning vectors, expression vectors, or both. The term vector and the term “plasmid” are used interchangeably.

The term “expression vector” or “expression plasmid” denotes the vehicle by which an expression cassette can be introduced into a host cell, so as to transform the host and promote expression (e.g., transcription and translation) of the introduced sequence.

The terms “express” and “expression” mean allowing or causing the information in a gene or DNA sequence to become manifest, for example, producing a protein by activating the cellular functions involved in transcription and translation of a corresponding gene or DNA sequence. A DNA sequence is expressed in or by a cell to form an “expression product” such as a protein. The expression product itself, for example, the resulting protein, may also be said to be “expressed.” An expression product can be characterized as intracellular, extracellular, or secreted.

An “amino acid modification” denotes a change in the amino acid sequence of a predetermined amino acid sequence. Exemplary modifications include an amino acid substitution, insertion and/or deletion.

An “amino acid insertion” refers to the incorporation of at least one amino acid into a predetermined amino acid sequence. An insertion may consist of the insertion of one or two amino acid residues or larger insertions. The inserted residue(s) may be naturally occurring or non-naturally occurring as disclosed above.

An “amino acid deletion” refers to the removal of at least one amino acid residue from a predetermined amino acid sequence.

An “amino acid substitution” refers to the replacement of at least one existing amino acid residue in a predetermined amino acid sequence with another different “replacement” amino acid residue. The replacement residue or residues may be “naturally occurring amino acid residues” (i.e., encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); Isoleucine (Ile): leucine (Leu); lysine (Lys); methionine (Met); phenylalanine (Phe); proline (Pro): serine (Ser); threonine (Thr); tryptophan (Trp); tyrosine (Tyr); and valine (Val). Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine, and other amino acid residue analogues such as those described in Ellman, et al. (Meth. Enzym. 202:301-336, 1991). To generate such non-naturally occurring amino acid residues, the procedures of Noren, et al. (Science 244:182, 1989) and Ellman, et al., 1991 may be used. Briefly, these procedures involve chemically activating a suppressor tRNA with a non-naturally occurring amino acid residue followed by in vitro transcription and translation of the RNA. Finally, one of skill in the art will recognize that an amino acid substitution of, for example, a region of a protein could be achieved in one step, or in two steps (e.g., by an amino acid deletion followed by an amino acid insertion or vice versa).

A “variant” of a specified polypeptide or protein comprises an amino acid sequence which differs from that of the specified polypeptide or protein by virtue of at least one “amino acid modification” as herein defined. A “variant” includes fragments of the polypeptide or protein that exhibit the desired activity, such as fragments of the Fc region of IgG that bind to FcRn and thereby improve circulating half-life when coupled to a coagulation factor.

“Fusion polypeptide” denotes a polypeptide comprising at least two discrete peptide portions which are not found to naturally occur in the same polypeptide.

“Fusion protein” denotes a protein comprising at least one fusion polypeptide. Thus, a multi-subunit protein is denoted as a fusion protein even if only one of its subunits is a fusion polypeptide.

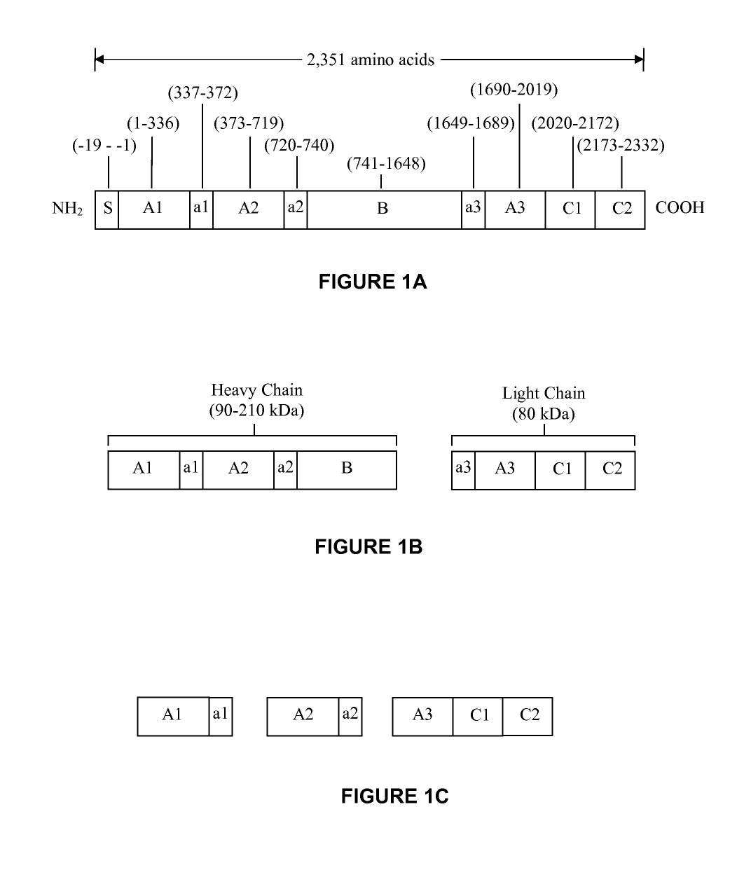

The terms “FVIII,” “Factor VIII,” or “Factor VIII protein” are intended to encompass a wild-type Factor VIII protein, including functional allelic variants, or any derivative, variant, or analogue thereof, which possesses the biological activity of Factor VIII. For purposes of this definition, “biological activity of Factor VIII” refers to its ability to participate in the intrinsic pathway of blood coagulation. Generally, this biological activity may be determined with reference to a Factor VIII standard derived from plasma using a commercially available Factor VIII assay (Coatest®, diaPharma®, West Chester, Ohio) or other assay in the art.

Where reference is made to a Factor VIII domain, “domain” is used to denote the approximate regions of Factor VIII known to those skilled in the art. With respect to human Factor VIII, the amino acid numbering for the different Factor VIII domains is shown in FIG. 1.

“Factor VIII fusion gene” denotes a non-naturally occurring nucleic acid construct which codes for a “Factor VIII fusion protein” as defined further below and which may be produced by operative insertion of nucleic acid coding for a modulator into nucleic acid coding for a Factor VIII protein at a position within the Factor VIII protein coding sequence corresponding to the B domain coding portion. As an example, at least a portion of the B domain coding sequence may be deleted and replaced by the nucleic acid coding for the modulator. As will be appreciated by one of skill in the art, “operative insertion” is only intended to encompass those insertions of nucleic acid coding for a modulator which produce a nucleic acid construct in which the portion of the nucleic acid coding for the modulator and the nucleic acid coding for the portions of Factor VIII that are upstream and downstream of the nucleic acid coding for the modulator are all in proper reading frame. A Factor VIII fusion gene may further comprise additional nucleic acid sequences coding for a peptide linker or multimerization sequence. Finally, for purposes of the above definition, “gene” is not intended to imply the presence of any nucleic acid sequence which would otherwise be required to enable transcription, translation, or proper post-translational processing (i.e., promoter, enhancers, signal peptides, secretory leader sequences, etc.).

“Factor VIII fusion protein” denotes the full length polypeptide produced by transcription and translation of a Factor VIII fusion gene, but which has not yet undergone post-translational processing. During post-translational processing, a Factor VIII fusion protein is converted to a “Factor VIII fusion heterodimer” as defined below.

“Factor VIII fusion heterodimer” denotes a heterodimeric protein which has the biological activity of Factor VIII and which is produced as a result of transcription and translation of a Factor VIII fusion gene, and post-translational modification (including proteolytic processing) of the Factor VIII fusion protein produced thereby. Thus, a Factor VIII fusion heterodimer is analogous to the heterodimeric form of wild-type Factor VIII which is found circulating in blood plasma (i.e., comprising a heavy chain and light chain). A Factor VIII fusion heterodimer of the present invention may differ from the heterodimeric form of the Factor VIII protein from which it is derived in that it is comprised of, for example, a “modified heavy chain” (i.e., a Factor VIII heavy chain which comprises a modulator and may also have deletions of at least a portion of the B-domain). The Factor VIII fusion heterodimers of the present invention may exhibit, for example, increased circulating half-life in comparison to the Factor VIII protein from which the fusion heterodimer is derived. “Factor VIII fusion heterodimer(s)” may also encompass “multimeric” and “hybrid” Factor VIII fusion heterodimers as defined further below.

“Multimeric Factor VIII fusion heterodimer” denotes proteins comprising at least two Factor VIII fusion heterodimers. Multimeric Factor VIII fusion heterodimers may arise if an amino acid, peptide, or polypeptide portion of the modulator present in a first Factor VIII fusion heterodimer is capable of mediating a non-covalent or covalent association with a homologous or heterologous portion of a modulator present in a second Factor VIII fusion heterodimer. For example, the hinge region of the Fc portion of IgG is capable of mediating covalent association between two Factor VIII fusion heterodimers, regardless of whether the Factor VIII fusion heterodimers have identical amino acid sequences (in which case the multimeric Factor VIII fusion heterodimer could be referred to as a “homo-multimeric Factor VIII fusion heterodimer”) or different amino acid sequences (in which case the multimeric Factor VIII fusion heterodimer could be referred to as a “hetero-multimeric Factor VIII fusion heterodimer”). Multimeric Factor VIII fusion heterodimers may also arise if in addition to nucleic acid coding for a modulator, a Factor VIII fusion gene comprises an operatively linked nucleic acid coding for an amino acid, peptide, or polypeptide capable of mediating a non-covalent or covalent association with a homologous amino acid, peptide, or polypeptide (hereafter denoted as a “homo-multimerization sequence”) or heterologous peptide or polypeptide (hereafter denoted as a “hetero-multimerization sequence”). The skilled artisan will appreciate that a second distinct Factor VIII fusion gene may be required to produce a multimeric Factor VIII fusion heterodimer when the first Factor VIII fusion gene only contains a hetero-multimerization sequence. For example, the skilled artisan would recognize that in order to utilize the “protuberance-into-cavity” approach described in U.S. Pat. No. 5,807,706, two Factor VIII fusion genes would be required. With regard to recombinant production of multimeric Factor VIII fusion heterodimers, the skill artisan will appreciate that homo-multimeric forms may be produced by a single recombinant host cell, whereas hetero-multimeric forms may be produced by co-expression within a single host cell or separate expression in multiple host cells (in the same or different cell culture systems). While not intending to be limited to currently known approaches, the general approaches for producing multimeric polypeptides taught in the following non-limiting references could be adapted for use in producing a multimeric Factor VIII fusion heterodimer: US Patent Application Publication No. 2007/0287170; the “multimerization domain” approaches disclosed in U.S. Pat. No. 7,183,076, for example, those employing immunoglobulin moieties; use of Fos and Jun leucine zippers as employed in U.S. Pat. No. 5,932,448; and the “heterodimerization sequence” approach employed in U.S. Pat. No. 6,833,441.

“Hybrid Factor VIII fusion heterodimer” denotes any recombinant protein of the invention comprising only a single Factor VIII fusion heterodimer which is covalently or non-covalently associated with at least one other polypeptide. The skilled artisan will appreciate that where a modulator is capable of forming a dimer or multimer (e.g., the dimeric Fc region of an immunoglobulin), it is possible to produce a multimeric Factor VIII fusion heterodimer (as defined above). However, the skilled artisan will appreciate that not every polypeptide of a multimeric half-life modulator needs to be expressed as a Factor VIII fusion protein. For example, where an Fc region is used as the modulator, an expression cassette coding for only an Fc region (or an Fc region operatively linked to an affinity tag or non-Factor VIII peptide, protein or protein fragment) may be introduced into the same or different host cell comprising an expression cassette comprising a Factor VIII fusion gene. The skilled artisan will also appreciate that a hybrid Factor VIII fusion heterodimer may be designed even when the modulator is incapable of forming a dimer or multimer. Specifically, a homo- or heterodimer sequence may be positioned within a Factor VIII fusion gene either 5′ (N-terminal in relationship to when expressed) or 3′ (i.e., C-terminal in relationship to when expressed) to the nucleic acid coding for the modulator.

The term “modulator” refers to any polypeptide, protein, protein fragment(s), or a variant thereof (comprised of one or more polypeptide subunits), which when inserted or substituted into a protein (e.g., Factor VIII) modifies, for example, the activity and/or pharmacokinetic properties of the protein. As an example, “half-life modulator” may increase or decrease the circulating half-life of a protein (e.g., Factor VIII fusion heterodimer, hybrid Factor VIII fusion heterodimer, or multimeric Factor VIII fusion heterodimer produced as a result of said insertion or substitution) in comparison to the protein from which it is derived. A half-life modulator may, for example, increase the circulating half-life of a protein (e.g., Factor VIII fusion heterodimer, hybrid Factor VIII fusion heterodimer, or multimeric Factor VIII fusion heterodimer) by at least 10%, by at least 20%, by at least 30% or by at least 40%, by at least 50%, by at least 60%, by at least 70%, by at least 80%, by at least 90%, or by at least 100%. In one embodiment, the half-life modulator may increase the circulating half-life of a Factor VIII fusion heterodimer, hybrid Factor VIII fusion heterodimer, or multimeric Factor VIII fusion heterodimer at least about twofold in comparison with the Factor VIII protein from which it is derived, and in further embodiments increase the circulating half-life at least about 2.5-fold, at least about threefold, or more. In another embodiment, a half-life modulator does not include any endogenous elements of a Factor VIII protein, such as, without limitation, the B-domain.

The term “circulating half-life,” “plasma half-life,” “serum half-life,” or “t [½]” as used herein in the context of administering a peptide drug to a patient, may be defined as the time required for plasma concentration of a drug in a patient to be reduced by one half. There may be more than one half-life associated with the peptide drug depending on multiple clearance mechanisms, redistribution, and other mechanisms well known in the art. Usually, alpha, and beta half-lives are defined such that the alpha phase is associated with redistribution, and the beta phase is associated with clearance. However, with protein drugs that are, for the most part, confined to the bloodstream, there can be at least two clearance half-lives. For purposes of the present invention, beta half life may be calculated by measuring plasma protein levels (using, for example, antigen ELISA) at suitably selected timepoints following administration, or by measuring coagulant activity (using, for example, a Coatest assay) at suitably selected timepoints. Further explanation of “half-life” may be found in Pharmaceutical Biotechnology (1997, D F A Crommelin and R DSindelar, eds., Harwood Publishers, Amsterdam, pp 101-120).

### Construction of Factor VIII Fusion Genes

One aspect of the present invention relates to a Factor VIII fusion gene. The Factor VIII fusion gene can be either RNA or DNA. As noted previously, a Factor VIII fusion gene is a nucleic acid molecule that codes for a Factor VIII fusion protein. A Factor VIII fusion gene is derived from a Factor VIII coding sequence, nucleic acid coding for a modulator, and optionally, nucleic acid coding for a homo- or hetero-multimerization sequence which is distinct from the nucleic acid coding for a modulator. While these components of a Factor VIII fusion gene are detailed further below, the skilled artisan will appreciate that construction of a Factor VIII fusion gene can be synthesized from nucleic acid coding for these discrete components using well-known procedures. A variety of methods that may find use in the present invention are described in Molecular Cloning—A Laboratory Manual, 3rd Ed. (Maniatis, Cold Spring Harbor Laboratory Press, New York, 2001), and Current Protocols in Molecular Biology (John Wiley & Sons).

### Selection of Nucleic Acid Coding for Factor VIII

The recombinant Factor VIII fusion proteins and heterodimers of the present invention may be prepared by modifying nucleic acid which codes for a wild-type Factor VIII, a natural allelic variant of Factor VIII that may exist and occur from one individual to another, a chimeric Factor VIII (e.g., human/porcine), or a mutant factor VIII that has otherwise been modified yet retains procoagulant function, such as mutants that have been modified to affect properties of a wild-type Factor VIII or Factor VIIIa protein, such as glycosylation sites and patterns, antigenicity, specific activity, circulating half-life, protein secretion, affinity for factor IXa and/or factor X, altered factor VIII-inactivation cleavage sites, stability of the activated Factor VIIIa form, immunogenicity, shelf-life, etc. Suitable mutant Factor VIII sequences that may be modified in accordance with the present invention may include any previously known or subsequently identified variant Factor VIII sequences that have the procoagulant function associated with wild-type Factor VIII.

Suitable wild-type Factor VIII that can be modified in accordance with the present invention can be from various animals including, without limitation, mammals such as humans (see, e.g., GenBank Accession Nos. AAA52484 (amino acid) (SEQ ID NO: 1) and K01740 (nucleotide) (SEQ ID NO: 2), GenBank Accession Nos. AAA52485 (amino acid) (SEQ ID NO:3) and M14113 (nucleotide) (SEQ ID NO:4), and GenBank Accession No. AAA52420 (amino acid) (SEQ ID NO:5)); rats (see, e.g., GenBank Accession Nos. AAQ21580 (amino acid) and AY362193 (nucleotide)); mice (see, e.g., GenBank Accession Nos. AAA37385 (amino acid) and L05573 (nucleotide)); dogs (see, e.g., GenBank Accession Nos. AAB87412 (amino acid) and AF016234 (nucleotide)); bats (see, e.g., GenBank Accession Nos. ACC68917 (amino acid) and DP000725 (nucleotide)); chickens (see, e.g., GenBank Accession Nos. AAO33367 (amino acid) and AF465272 (nucleotide)); chimpanzees (see, e.g., GenBank Accession Nos. XP_{—}529212 (amino acid) and XM_{—}529212 (nucleotide)); pigs (see, e.g., GenBank Accession Nos. NP_{—}999332 (amino acid) and NM_{—}214167 (nucleotide)); rabbits (see, e.g., GenBank Accession Nos. ACA42556 (amino acid) and EU447260 (nucleotide)); cats, monkeys, guinea pigs, orangutans, cows, horses, sheep, goats, or other mammalian species. Sequences for human, porcine, murine' and canine are also available electronically via the Haemophilia A Mutation, Structure, Test and Resource Site (or HAMSTeRS), which further provides an alignment of human, porcine, murine, and canine Factor VIII proteins. As one of skill in the art will appreciate, the conservation and homology among mammalian Factor VIII proteins is well known.

One non-limiting example of a suitable mutant Factor VIII that may be modified in accordance with the present invention is a B-domain deleted Factor VIII (“BDD Factor VIII”) characterized by having the amino acid sequence which contains a deletion of all but 14 amino acids of the B-domain (SFSQNPPVLKRHQR, SEQ ID NO: 6) of naturally occurring human FVIII. (Lind, et al., Eur. J. Biochem. 232:19-27, 1995). This BDD Factor VIII has the amino acid sequence of SEQ ID NO:7.

Another non-limiting example of a suitable mutant Factor VIII that may be modified in accordance with the present invention is a chimeric human/animal Factor VIII that contains one or more animal amino acid residues as substitution(s) for human amino acid residues that are responsible for the antigenicity of human Factor VIII (see, e.g., U.S. Pat. Nos. 5,364,771; 5,663,060; and 5,888,974). For example, animal (e.g., porcine) residue substitutions can include, without limitation, one or more of the following: R484A, R488G, P485A, L4865, Y487L, Y487A, S488A, S488L, R489A, R489S, R490G, L491S, P492L, P492A, K493A, G494S, V495A, K496M, H497L, L4985, K499M, D500A, F501A, P502L, 1503M, L504M, P505A, G506A, E507G, 1508M, 1508A, M2199I, F2200L, L2252F, V2223A, K2227E, and/or L2251 (see, e.g., U.S. Pat. Nos. 5,859,204 and 6,770,744 and US Patent Application Publication No. 2003/0166536).

Another non-limiting example of a suitable mutant Factor VIII that may be modified in accordance with the present invention is a Factor VIII that is characterized by greater stability of activated Factor VIII by virtue of fused A2 and A3 domains. For example, a Factor VIII may be modified by substituting cysteine residues at positions 664 and 1826, resulting in a mutant factor VIII that includes a Cys664-Cys1826 disulfide bond that covalently links the A2 and A3 domains (Gale, et al., J. Thromb. Haemost. 1:1966-1971, 2003).

An additional non-limiting example of a suitable mutant Factor VIII that may be modified in accordance with the present invention is a Factor VIII with altered inactivation cleavage sites (see, e.g., Amano, et al., Thromb. Haemost. 79:557-63, 1998; Thornburg, et al., Blood 102:299, 2003). These alterations may be used to decrease a mutant Factor VIII's susceptibility to cleavage enzymes that normally inactivate the wild type Factor VIII.

Another non-limiting example of a suitable mutant Factor VIII that may be modified in accordance with the present invention is a Factor VIII that has enhanced affinity for Factor IXa (see, e.g., Fay, et al., J. Biol. Chem. 269:20522-20527, 1994); Bajaj, et al., J. Biol. Chem. 276:16302-16309, 2001; and Lenting, et al., J. Biol. Chem. 271:1935-1940, 1996) and/or Factor X (see, e.g., Lapan, et al., J. Biol. Chem. 272:2082-2088, 1997).

Another non-limiting example of a suitable mutant Factor VIII that may be modified in accordance with the present invention is a Factor VIII that is modified to enhance secretion of the Factor VIII (see, e.g., Swaroop, et al., J. Biol. Chem. 272:24121-24124, 1997).

An additional non-limiting example of a suitable mutant Factor VIII that may be modified in accordance with the present invention is a Factor VIII with an increased circulating half-life. These mutant Factor VIII proteins can be characterized as having, without limitation, reduced interactions with heparan sulfate (Sarafanov, et al., J. Biol. Chem. 276:11970-11979, 2001) and/or reduced interactions with low-density lipoprotein receptor-related protein (“LRP”) (see, e.g., WO 00/28021; WO 00/71714; Saenko, et al., J. Biol. Chem. 274:37685-37692, 1999; and Lenting, et al., J. Biol. Chem. 274:23734-23739, 1999).

Another non-limiting example of a suitable mutant Factor VIII that may be modified in accordance with the present invention is a modified Factor VIII encoded by a nucleotide sequence modified to code for amino acids within known, existing epitopes to produce a recognition sequence for glycosylation at asparagines residues (see, e.g., U.S. Pat. No. 6,759,216). The mutant Factor VIII of this example may be useful in providing a modified Factor VIII that escapes detection by existing inhibitory antibodies (low antigenicity Factor VIII) and which decreases the likelihood of developing inhibitory antibodies (low immunogenicity Factor VIII). In one embodiment of this type of mutant Factor VIII which may be modified in accordance with the present invention is a Factor VIII which is mutated to have a consensus amino acid sequence for N-linked glycosylation. An example of such a consensus sequence is N—X—S/T, where N is asparagine, X is any amino acid, and S/T stands for serine or threonine (see, e.g., U.S. Pat. No. 6,759,216).

Another non-limiting example of a suitable mutant Factor VIII that may be modified in accordance with the present invention is a procoagulant-active Factor VIII having various mutations (see, e.g., U.S. Pat. No. 6,838,437 and U.S. Patent Application Publication No. 2004/0092442). One example of this embodiment relates to a mutant Factor VIII that has been modified to (i) delete the von Willebrand factor binding site, (ii) add a mutation at Arg 740, and (iii) add an amino acid sequence spacer between the A2- and A3-domains, where the amino acid spacer is of a sufficient length so that upon activation, the procoagulant-active Factor VIII protein becomes a heterodimer (see, e.g., US Patent Application Publication No. 2004/0092442; Pittman, et al., PNAS 85:2429-2433, 1988: disclosing that cleavage at Arg740 is not essential to generate co-factor activity).

Another non-limiting example of a suitable mutant Factor VIII that may be modified in accordance with the present invention is a mutant Factor VIII which is encoded by a nucleotide sequence having a truncated factor IX intron 1 inserted in one or more locations (see, e.g., U.S. Pat. Nos. 6,800,461 and 6,780,614). This mutant Factor VIII may be used for yielding higher production of the recombinant Factor VIII in vitro as well as in a transfer vector for gene therapy (see, e.g., U.S. Pat. No. 6,800,461). In one example of this embodiment, the mutant Factor VIII may be encoded by a nucleotide sequence having a truncated factor IX intron 1 inserted in two locations, and having a promoter that is suitable for driving expression in hematopoietic cell lines and in platelets (see, e.g., U.S. Pat. No. 6,780,614).

An additional non-limiting example of a suitable mutant Factor VIII that may be modified in accordance with the present invention is a mutant Factor VIII which exhibits reduced inhibition by inhibitory antibodies (see, e.g., U.S. Pat. Nos. 5,859,204; 6,180,371; 6,458,563; and 7,122,634).

Another non-limiting example of a suitable mutant Factor VIII that may be modified in accordance with the present invention is a mutant Factor VIII which has one or more amino acid substitutions in the A2 domain which have the effect of increasing the half-life and/or specific activity of Factor VIII (see, e.g., U.S. Pat. No. 7,211,559).

An additional non-limiting example of a suitable mutant Factor VIII that may be modified in accordance with the present invention is a mutant Factor VIII which exhibits increased specific activity (see, e.g., US Patent Application Publication No. 2007/0265199).

Another non-limiting example of a suitable mutant Factor VIII that may be modified in accordance with the present invention is a FVIII mutein that has been covalently bound at a predefined site to one or more biocompatiable polymers (see, e.g., US Patent Application Publication No. 2006/0115876).

### Selection of Nucleic Acid Coding for a Modulator

The Factor VIII fusion genes of the present invention include nucleic acid encoding a modulator. For example, numerous proteins (and the nucleic acid encoding them) are known in the art which when fused with a therapeutic protein had the effect of extending the serum half-life in comparison to the unfused therapeutic protein. Nucleic acid encoding any of the modulators taught in these references may potentially be used for constructing a Factor VIII fusion gene of the present invention. Considerations for selecting candidate modulators which may, for example, potentially increase the circulating half-life of a Factor VIII fusion heterodimer (in comparison to the Factor VIII protein from which it is derived) include: (1) the circulating half-life of the modulator should be greater than the circulating half-life of the Factor VIII protein selected for modification; and (2) immunogenicity of the fusion protein. Regarding the second consideration, it may be preferable to use a modulator which is naturally expressed or derived from a protein which is naturally expressed in the population (e.g., humans) intended to be treated with the Factor VIII fusion heterodimer. For example, the modulator is naturally present in the serum of the population intended to be treated (e.g., use of a human Fc region where humans are the intended treatment population).

In one embodiment of the invention, immunoglobulin constant regions may be used as modulators. Accordingly, a modulator coding nucleic acid sequences used in constructing Factor VIII fusion genes of the invention may be polynucleotides encoding an Fc region of an immunoglobulin (Ig) or a fragment and/or variant thereof, and polynucleotides encoding a FcRn binding peptide or variant thereof. In one embodiment, the nucleic acid used codes for a modulator which is an Fc region or a fragment and/or variant thereof of an immunoglobulin obtained from human IgG1, IgG2, IgG3, IgG4, IgE, IgD, or IgM, or mouse IgG1, IgG2a, IgG2b, IgG3, IgA, or IgM. In another embodiment, the nucleic acid used codes for a modulator which is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG. In an additional embodiment, the nucleic acid used codes for a modulator which is an Fc region of a mouse IgG1 or a human IgG1, or the non-hinge portion of an Fc region of a human IgG1 or mouse IgG1. In a further embodiment, the nucleic acid used codes for a modulator which is an Fc region of a human IgG1, a variant Fc region of a human IgG1 which has a non-functional hinge (by substitution or deletion of cysteine(s) residues), the non-hinge portion of an Fc region of a human IgG1.

For the fragments of Fc regions of immunoglobulins, a nucleic acid which codes for a modulator may code for at least an amino acid segment of an Fc region which defines an epitope bound by a neonatal Fc receptor (FcRn), and may further code for a segment corresponding to the hinge portion of an Fc region. Alternatively, the nucleic acid which codes for a moduclator may code for at least a FcRn binding peptide. Without limitation, examples of suitable FcRn binding peptide include the sequence PKNSSMISNTP (SEQ ID NO:24) and may further include a sequence selected from HQSLGTQ (SEQ ID NO:25), HQNLSDGK (SEQ ID NO:26), HQNISDGK (SEQ ID NO:27), or VISSHLGQ (SEQ ID NO:28) (see, e.g., U.S. Pat. No. 5,739,277).

In one embodiment of the present invention, the modulators may be encoded by a nucleic acid sequence coding for an amino acid sequence identical to or sharing at least about 70%, at least about 75%, at least about 80%, at least about 85%, at least about 90%, or at least about 95% amino acid identity with SEQ ID NO: 9 (Fc region of a human IgG1), SEQ ID NO: 11 (Fc region of a human IgG2), SEQ ID NO: 13 (Fc region of a human IgG3), SEQ ID NO: 15 (Fc region of a human IgG4), SEQ ID NO: 29 (Fc region of a mouse IgG1), SEQ ID NO: 17 (non-hinge portion of the Fc region of a human IgG1), SEQ ID NO: 19 (non-hinge portion of the Fc region of a human IgG2), SEQ ID NO: 21 (non-hinge portion of the Fc region of a human IgG3), SEQ ID NO: 23 (non-hinge portion of the Fc region of a human IgG4), or SEQ ID NO: 30 (non-hinge portion of the Fc region of a mouse IgG1). Specific examples of nucleic acids which encode for one of the above include SEQ ID NO: 8 (Fc region of a human IgG1), SEQ ID NO: 10 (Fc region of a human IgG2), SEQ ID NO: 12 (Fc region of a human IgG3), SEQ ID NO: 14 (Fc region of a human IgG4), SEQ ID NO: 47 (Fc region of a mouse IgG1), SEQ ID NO: 16 (non-hinge portion of the Fc region of a human IgG1), SEQ ID NO: 18 (non-hinge portion of the Fc region of a human IgG2), SEQ ID NO: 20 (non-hinge portion of the Fc region of a human IgG3), SEQ ID NO: 22 (non-hinge portion of the Fc region of a human IgG4), and SEQ ID NO: 48 (non-hinge portion of the Fc region of a mouse IgG1).

### Method for Identifying of Nucleic Acid Coding for a Modulator

Other polypeptides or proteins may be identified as suitable modulators by use of the methodology described herein. Candidate modulators (e.g., polypeptides which may potentially be useful in creating Factor VIII fusion genes and Factor VIII fusion proteins), include those peptides and proteins which have been shown to extend the serum half-life of non-Factor VIII therapeutic proteins or peptides by fusion to the therapeutic protein. For example, one method for identifying modulators of a Factor VIII protein is to examine the pharmacokinetics of a Factor VIII fusion heterodimer comprising a modulator in a hemophilia A animal model, such as Hemophilia A (HemA) mice.

### Insertion Site For Nucleic Acid Coding for a Modulator

The Factor VIII fusion genes of the present invention include nucleic acid encoding a modulator. The nucleic acid encoding the modulator may be inserted within the B-domain portion of a Factor VIII gene. For example, at least a portion of the nucleic acid encoding the B-domain of a Factor VIII gene may be deleted prior to or subsequent to insertion of the nucleic acid encoding the modulator (e.g., delete at least the portion of the Factor VIII gene coding for the portion of the B domain from N-745 to S-1637). Alternatively, site-specific recombination may be used to simultaneously insert nucleic acid encoding a modulator and delete a portion of the B-domain region coding nucleic acid. Recombinant methods for achieving insertions, deletions, and site-specific recombinations are well known in the art.

In one embodiment, the Factor VIII fusion gene comprises a nucleic acid sequence encoding a Factor VIII fusion protein, wherein the Factor VIII fusion protein comprises a Factor VIII protein in which an amino acid sequence of a modulator is present in the B-domain, or an amino acid sequence of a modulator replaces some or all of the amino acid sequence of the B-domain. In a second embodiment, the Factor VIII fusion gene comprises a nucleic acid sequence encoding a Factor VIII fusion protein which comprises a first amino acid sequence corresponding to amino acids 20-764 of any one of SEQ ID NOS: 1 or 5, a second amino acid sequence corresponding to amino acids 1656-2351 of any one of SEQ ID NOS: 1 or 5, and a modulator amino acid sequence in which the half-life modulator amino acid sequence is covalently attached at its amino terminal to the carboxyl terminal of the first amino acid sequence and covalently attached at its carboxyl terminal to the amino terminal of the second amino acid.

Prior to secretion, the B domain is cleaved at Arg¹⁶⁴⁸ (i.e., the B-a3 junction) and variably cleaved in the B-domain, predominantly after Arg¹³¹³ (see, e.g., Thompson, Semin. Thromb. Hemost. 29:11-22, 2003). Thus, the skilled artisan will recognize that for insertions, deletions and/or substitutions in the B-domain region, the cleavage site occurring at the B-a3 domain junction should be maintained for proper post-translational processing of a Factor VIII fusion protein into a Factor VIII fusion heterodimer. Likewise, for insertions in the B-domain region, nucleic acid coding for a modulator should be inserted at a site within the nucleic acid coding for the B-domain which is 5′ to the nucleic acid coding for Arg¹³¹³. Alternatively, cleavage sites within the B-domain (with the exception of the cleavage site at the B-a3 junction) may be mutated to prevent cleavage (and therefore separation) of the modulator from the N-terminal (“heavy chain”) portion during post-translational processing of the Factor VIII fusion protein.

It is known that cleavage at the a2-B domain junction is not essential to generate co-factor activity of Factor VIII (Pittman, et al., PNAS 85:2429-2433, 1988). In human Factor VIII, the a2-B domain junction occurs at Arg740. Factor VIII fusion genes of the present invention include genes coding for Factor VIII fusion proteins which undergo cleavage at the a2-B domain junction as well as genes coding for Factor VIII fusion proteins which have an amino acid modification at the a2-B domain junction which prevents cleavage. As an example, the a2-B domain junction cleavage site may be left intact, as cleavage at this junction upon activation of the Factor VIII fusion heterodimers of the present invention results in formation of a Factor VIIIa protein identical to (and therefore having the same biological activity as) the Factor VIIIa protein which is produced upon activation of the Factor VIII protein from which the Factor VIII fusion heterodimer is derived.

One of skill in the art will appreciate that if degree or rate of cleavage at the a2-B domain junction in a Factor VIII fusion heterodimer is less than what is seen in the Factor VIII protein from which it is derived, it is most likely due to steric hindrance by the modulator. Thus, it may be desirable to include additional amino acids in the form of a peptide linker (i.e., spacer) between the a2-B domain junction and half-life modulator such as the peptide linkers DDDDK (SEQ ID NO: 49) and GGGGSGGGGSGGGGS (SEQ ID NO: 50).

### Selection and Insertion Site of Nucleic Acid Coding for a Homo- or Hetero-multimerization Sequence

The Factor VIII fusion genes of the present invention optionally include a nucleic acid coding for a homo- or hetero-multimerization sequence which is distinct from the nucleic acid coding for a modulator. Inclusion of nucleic acid coding for a homo- or hetero-multimerization sequence may be desired in order to produce a multimeric Factor VIII fusion heterodimer when the modulator employed in a first Factor VIII fusion heterodimer is not capable of mediating a non-covalent or covalent association with a homologous or heterologous portion of a modulator present in a second Factor VIII fusion heterodimer. Alternatively, inclusion of nucleic acid coding for a homo- or hetero-multimerization sequence may be desired in order to produce a hybrid Factor VIII fusion heterodimer when the modulator employed in a first Factor VIII fusion heterodimer consists of a single polypeptide.

As will be appreciated by one of skill in the art, selection of nucleic acid coding for a homo- or hetero-multimerization sequence will be dictated by the specific multimerization approach utilized. Nucleic acid sequences coding for the homo- or hetero-multimerization sequences employed in the general approaches for producing multimeric polypeptides taught in the following non-limiting references could be incorporated into the Factor VIII fusion genes of the present invention: US Patent Application Publication No. 2007/0287170; the “multimerization domain” approaches disclosed in U.S. Pat. No. 7,183,076, for example, those employing immunoglobulin moieties; use of Fos and Jun leucine zippers as employed in U.S. Pat. No. 5,932,448; and the “heterodimerization sequence” approach employed in U.S. Pat. No. 6,833,441; and the “protuberance-into-cavity” approach described in U.S. Pat. No. 5,807,706.

The skilled artisan will appreciate that a second distinct Factor VIII fusion gene may be required to produce a multimeric Factor VIII fusion heterodimer or hybrid Factor VIII fusion heterodimer when the first Factor VIII fusion gene only contains a hetero-multimerization sequence. For example, the skilled artisan would recognize that in order to utilize the “protuberance-into-cavity” approach described in U.S. Pat. No. 5,807,706, two Factor VIII fusion genes, each comprising a distinct hetero-multimerization sequence, would be required.

The skilled artisan will recognize that a nucleic acid coding for a homo- or hetero-multimerization sequence within a Factor VIII fusion gene may be positioned within a Factor VIII fusion gene either 5′ (i.e., N-terminal in relationship to when expressed) or 3′ (i.e., C-terminal in relationship to when expressed) to a modulator provided that its position does not interfere with transcription, translation, or post-translational modification which is otherwise required for formation of a Factor VIII fusion heterodimer. The nucleic acid coding for the multimerization sequence, like the nucleic acid coding for a modulator, may be inserted within or replaces at least a portion of the region of a Factor VIII gene coding for the B domain.

### Expression Cassettes and Expression Vectors

A further aspect of the present invention relates to an expression cassette or expression vector comprising a Factor VIII fusion gene. For recombinant production of an expression cassette comprising a Factor VIII fusion gene, a Factor VIII fusion gene is isolated and operatively linked to a promoter. The Factor VIII fusion gene may optionally be further operatively linked to transcription termination signals, nucleic acid coding for signal peptides, or other nucleic acid sequences that influence gene expression or postranslation processing (e.g., conveniently located restriction sites, enhancers, secretory leader sequences, etc.). If the desired components of an expression cassette (other than a Factor VIII fusion gene) are already contained within a replicable cloning vector or expression vector, then a Factor VIII fusion gene need only be operatively inserted in the proper location by recombinant techniques well known in the art. Many cloning vectors are commercially available and generally include one or more of the following: a signal sequence, an origin of replication, an enhancer element, a promoter, transcription termination sequence, and one or more selection genes or markers. Many expression vectors are also commercially available and insertion of a Factor VIII fusion gene may be accomplished using methods and reagents that are well known in the art (see, e.g., Sambrook, et al., Molecular Cloning: A Laboratory Manual, Second Edition, Cold Spring Harbor Press, NY (1989); Ausubel, et al., Current Protocols in Molecular Biology, New York, N.Y.: John Wiley & Sons (1989). The selection of an expression vector will depend on the preferred transformation technique and target host for transformation.

Expression vectors useful in the present invention include, but are not limited to, chromosomal-, episomal- and virus-derived vectors, for example, vectors derived from bacterial plasmids, bacteriophages, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids. Suitable viral vectors for recombinant expression in animal cells are well known in the art (see, e.g., U.S. Pat. Nos. 5,871,986 and 6,448,046).

Suitable vectors for practicing the present invention include, but are not limited to, the following viral vectors such as lambda vector system gt11, gtWES.tB, Charon 4, and plasmid vectors such as pCMV, pBR322, pBR325, pACYC177, pACYC184, pUC8, pUC9, pUC18, pUC19, pLG339, pR290, pKC37, pKC101, SV 40, pBluescript II SK+/− or KS+/− (Stratagene, LaJolla, Calif.), pQE, pIH821, pGEX, pET series (Studier, et al., Methods Enzymol. 185:60-89, 1990), and any derivatives thereof. Suitable vectors for use in bacteria include pQE70, pQE60, and pQE-9 (Qiagen, Valencia, Calif.); pBS vectors, Phagescript vectors, Bluescript vectors, pNH8A, pNH16a, pNH18A, and pNH46A (Stratagene, LaJolla, Calif.); pcDNA3 (Invitrogen, Carlsbad, Calif.); and pGEX, ptrxfus, ptrc99a, pET-5, pET-9, pKK223-3, pKK233-3, pDR540, and pRIT5. Suitable eukaryotic vectors are pWLNEO, pSV2CAT, pOG44, pXT1, pBK, and pSG (Stratagene, LaJolla, Calif.); and pSVK3, pBPV, pMSG, and pSVL. Other suitable vectors will be readily apparent to the skilled artisan.

An expression vector is used which comprises a gene coding for a selectable marker which confers a selectable phenotype such as drug resistance, nutritional auxotrophy, resistance to a cytotoxic agent or expression of a surface protein. Examples of selectable marker genes which can be used include neo, gpt, dhfr, ada, pac (puromycin), hyg, and hisD.

Successful ligations (or insertion into a vector) of a Factor VIII fusion gene may readily be determined by recombinant techniques well known in the art (e.g., isolation and sequencing using conventional procedures or use of oligonucleotide probes that are capable of binding specifically to linkage sites).

### Host Cells

A further aspect of the present invention relates to a host cell comprising a Factor VIII fusion gene. The Factor VIII fusion gene may be present within a cloning vector, an expression vector, or integrated in the host cell genome. In one embodiment, a host cell contains the necessary nucleic acid constructs in DNA molecule form, either as a stable plasmid or as a stable insertion or integration into the host cell genome. In another embodiment, the host cell can contain a DNA molecule in an expression system.

In one embodiment, a Factor VIII fusion gene of the present invention is incorporated into an appropriate vector in the sense direction, such that the open reading frame is properly oriented for the expression of the encoded protein under control of a promoter of choice. This involves the inclusion of the appropriate regulatory elements into the expression vector. These may include, for example, non-translated regions of the vector, useful promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used. A constitutive promoter is a promoter that directs expression of a gene throughout the development and life of an organism. An inducible promoter is a promoter that is capable of directly or indirectly activating transcription of one or more DNA sequences or genes in response to an inducer. In the absence of an inducer, the DNA sequences or genes will not be transcribed.

An expression vector of the present invention may be also include an operable 3′ regulatory region, selected from among those which are capable of providing correct transcription termination and polyadenylation of mRNA for expression in the host cell of choice, operatively linked to a DNA molecule which encodes for a protein of choice.

To recombinantly produce a Factor VIII fusion heterodimer in a host cell, a Factor VIII fusion gene may be incorporated into a host cell. Cloning vectors, expression vectors and plasmids may be introduced into cells via, for example, transformation, transduction, conjugation, mobilization, or electroporation, using recombinant techniques well known in the art.

Host cells may include, without limitation, mammalian cells, bacterial cells (e.g., E. coli), insect cells (e.g., Sf9 cells), fungal cells, yeast cells (e.g., Saccharomyces or Schizosaccharomyces), plant cells (e.g., Arabidopsis or tobacco cells), or algal cells. Mammalian cells suitable for carrying out the present invention include without limitation COS (e.g., ATCC No. CRL 1650 or 1651), baby hamster kidney (“BHK”) (e.g., ATCC No. CRL 6281), Chinese Hamster Ovary (“CHO”) (ATCC No. CCL 61), HeLa (e.g., ATCC No. CCL 2), 293 (ATCC No. 1573), NSO myeloma, CHOP, NS-1, and HKB11 (see, e.g., U.S. Pat. No. 6,136,599).

Suitable expression vectors for directing expression in mammalian cells generally include a promoter, as well as other transcription and translation control sequences known in the art. Common promoters include SV40, MMTV, metallothionein-1, adenovirus Ela, CMV, immediate early, immunoglobulin heavy chain promoter and enhancer, and RSV-LTR. One of skill in the art can readily select appropriate mammalian promoters based on their strength as a promoter. Alternatively, an inducible promoter can be employed for purposes of controlling when expression or suppression of a particular protein is desired. One of skill in the art can readily select appropriate inducible mammalian promoters from those known in the art.

Regardless of the host cell selected for recombinant production of Factor VIII fusion heterodimers of the present invention, increased protein expression may be achieved by replacing non-common codons in a Factor VIII fusion gene with more common codons (see, e.g., U.S. Pat. No. 6,924,365). The skilled artisan will appreciate that determining whether a particular Factor VIII fusion gene codon is “non-common” or “common” depends on the particular codon usage of the host cell selected for recombinant production.

### Production of Factor VIII Fusion Proteins and Heterodimers

In view of the recombinant technology discussed herein, another aspect of the present invention relates to a method of producing a Factor VIII fusion heterodimer of the present invention. This method involves growing a host cell of the present invention under conditions whereby the host cell expresses the Factor VIII fusion protein. Following post-translational modification of the Factor VIII fusion protein, recombinant Factor VIII fusion heterodimer may then purified and isolated. One aspect of the invention is a method for producing a Factor VIII fusion protein or Factor VIII fusion heterodimer comprising (a) providing a host cell transformed with an expression vector encoding the Factor VIII fusion protein or Factor VIII fusion heterodimer; (b) culturing the cell; and (c) isolating the Factor VIII fusion protein or Factor VIII fusion heterodimer. In a further embodiment, the host cell may be a mammalian host cell and the amino acid sequence of the modulator may be glycosylated.

With regard to recombinant production of multimeric Factor VIII fusion heterodimers, the skill artisan will appreciate that homo-multimeric forms may be produced by a single recombinant host cell, whereas hetero-multimeric forms may be produced by co-expression within a single host cell or separate expression in multiple host cells (in the same or different cell culture systems). Similar to hetero-multimeric forms, the skilled artisan will appreciate that hybrid Factor VIII fusion heterodimers may be produced by co-expression within a single host cell or separate expression in multiple host cells (in the same or different cell culture systems). Where separate cultures systems are utilized, the recombinant protein product from each culture may be isolated and then reassociated using standard techniques well known in the art. For recombinant production of multimeric Factor VIII fusion heterodimers and hybrid Factor VIII fusion heterodimers, a host cell may be selected that is capable of assembling the chains of the multimeric or hybrid Factor VIII fusion heterodimer in the desired fashion.

As an alternative to co-expression of separate genes, a monocistronic gene which encodes all of the needed polypeptide chains may be produced. For a particular example of how such a gene may be designed, see Example 5 below.

The recombinant Factor VIII fusion heterodimer may be produced in a substantially pure form. Methods well known in the art may used for the purification and identification of purified Factor VIII fusion heterodimer.

### Pharmaceutical Compositions

Another aspect of the present invention relates to a pharmaceutical composition comprising a Factor VIII fusion heterodimer and a pharmaceutically acceptable carrier. “Pharmaceutically acceptable carrier” is a substance that may be added to the active ingredient to help formulate or stabilize the preparation and causes no significant adverse toxicological effects to the patient. Examples of such carriers are well known to those skilled in the art and include water, sugars such as maltose or sucrose, albumin, salts such as sodium chloride, etc. Other carriers are described, for example, in Remington's Pharmaceutical Sciences by E. W. Martin. Such compositions will contain an effective amount of at least one Factor VIII fusion heterodimer.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. The composition may be formulated for parenteral injection. The composition may be formulated as a solution, microemulsion, liposome, or other ordered structure suitable to high drug concentration. The carrier may be a solvent or dispersion medium containing, for example, water, ethanol, polyol (e.g., glycerol, propylene glycol, and liquid polyethylene glycol, and the like), and suitable mixtures thereof. The composition may include isotonic agents, for example, sugars, polyalcohols such as mannitol, sorbitol, or sodium chloride. Examples of pharmaceutical compositions of Factor VIII are disclosed, for example, in U.S. Pat. Nos. 5,047,249; 5,656,289; 5,665,700; 5,690,954; 5,733,873; 5,919,766; 5,925,739; 6,835,372; and 7,087,723.

Sterile injectable solutions may be prepared by incorporating the active compound in the required amount in an appropriate solvent with one or a combination of ingredients enumerated above, as required, followed by sterilization microfiltration. Generally, dispersions may be prepared by incorporating the active compound into a sterile vehicle that contains a basic dispersion medium and the required other ingredients. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof.

### Methods of Treatment

Another aspect of the present invention relates to a method of treating genetic and acquired deficiencies in coagulation such as hemophilia (e.g., hemophilia A). This method involves administering to a patient exhibiting hemophilia A an effective amount of the Factor VIII fusion heterodimer (including hybrid or multimeric forms) of the present invention, whereby the patient exhibits effective blood clotting following vascular injury. A suitable effective amount of the Factor VIII fusion heterodimer consists of, without limitation, between about 10 to about 50 international units/kg body weight. The patient may be any mammal (e.g., a human).

The Factor VIII fusion heterodimers of the present invention may be administered intravenously, subcutaneously, or intramuscularly. Certain modulators may allow for oral administration.

The Factor VIII fusion heterodimers of the present invention may be used to treat uncontrolled bleeding due to Factor VIII deficiency (e.g., intraarticular, intracranial, or gastrointestinal hemorrhage) in hemophiliacs with and without inhibitory antibodies and in patients with acquired Factor VIII deficiency due to the development of inhibitory antibodies. In one embodiment, Factor VIII fusion heterodimer, alone, or in the form of a pharmaceutical composition (i.e., in combination with stabilizers, delivery vehicles, and/or carriers) is infused into patients intravenously according to the same procedure that is used for infusion of human or animal Factor VIII.

Alternatively, or in addition thereto, Factor VIII fusion heterodimers may be administered by administering a viral vector such as an adeno-associated virus which comprises a Factor VIII fusion gene expression construct (see, e.g., Gnatenko, et al., Br. J. Haematol. 104:27-36, 1999), or by transplanting cells genetically engineered to produce Factor VIII fusion heterodimer, typically via implantation of a device containing such cells. Such transplantation may involve using recombinant dermal fibroblasts (see, e.g., Roth, et al., New Engl. J. Med. 344:1735-1742, 2001); bone marrow stromal cells (see, e.g., U.S. Pat. No. 6,991,787), or hematopoietic progenitor host cells (see, e.g., U.S. Pat. No. 7,198,950). Viral vectors suitable for use in hemophilia A gene therapy (using nucleic acid coding for Factor VIII) and use thereof in gene therapy are known in the art (see, e.g., U.S. Pat. Nos. 6,200,560; 6,544,771; 6,649,375; 6,697,669; 6,773,709; 6,797,505; 6,808,905; 6,818,439; 6,897,045; 6,939,862; 7,198,950; and 7,238,346.)

The treatment dosages of Factor VIII fusion heterodimer that should be administered to a patient in need of such treatment will vary depending on the severity of the Factor VIII deficiency. Generally, dosage level is adjusted in frequency, duration, and units in keeping with the severity and duration of each patient's bleeding episode. Accordingly, Factor VIII fusion heterodimer may included in a pharmaceutically acceptable carrier, delivery vehicle, or stabilizer in an amount sufficient to deliver to a patient a therapeutically effective amount of the protein to stop bleeding, as measured by standard clotting assays.

Usually, the desired plasma Factor VIII activity level to be achieved in a patient through administration of the Factor VIII fusion heterodimers is in the range of 30-100% of normal. In one embodiment, administration of the therapeutic Factor VIII fusion heterodimers may be given intravenously at a dosage in the range from about 5 to about 50 units/kg body weight, in a range of about 10 to about 50 units/kg body weight, and at a dosage of about 20 to about 40 units/kg body weight; the interval frequency may be in the range from about 8 to 24 hours (in severely affected hemophiliacs); and the duration of treatment in days may be in the range from 1 to 10 days or until the bleeding episode is resolved or the administration of the Factor VIII fusion heterodimers may be prophylactic (see, e.g., Roberts, et al., pp 1453-1474, 1460, in Hematology, Williams, W. J., et al., ed. (1990)). As in treatment with human or plasma-derived Factor VIII, the amount of therapeutic recombinant Factor VIII infused may be defined by the one-stage Factor VIII coagulation assay and, in selected instances, in vivo recovery may determined by measuring the Factor VIII in the patient's plasma after infusion. It is to be understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed Factor VIII fusion heterodimers.

Treatment may take the form of a single administration or periodic or continuous administration over an extended period of time, as required or treatment may be administered for prophylactic purposes.

Factor VIII fusion heterodimers of the present invention exhibit increased circulating half-life in comparison to the Factor VIII protein from which they were derived. Factor VIII proteins having greater circulating half-life are useful in treatment of hemophilia because less frequent dosing will be required to correct a patient's Factor VIII deficiency. This increase in ease of administration may improve patient compliance with treatment protocol and thereby reduce the symptoms of coagulation disorders. Also, the reduced frequency of administration is expected to reduce the likelihood of developing an immune response to the Factor VIII because less antigen is administered.

The above disclosure generally describes the present invention. A more complete understanding may be obtained by reference to the following examples, which are provided for purposes of illustration only and are not intended to limit the scope of the invention.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

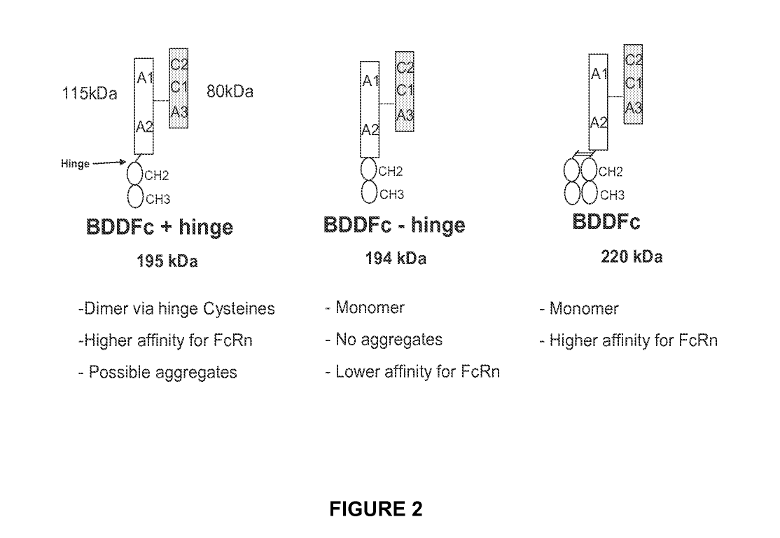

The following example describes construction of a mammalian expression vector (denoted as “pM110” or “pSK207BDDFc+hinge”) comprising a Factor VIII fusion gene (denoted as “BDDmFc+hinge”) using nucleic acid coding for a Factor VIII B-domain deleted (BDD) protein and nucleic acid coding for a murine Fc region (denoted as “mFc+hinge”). Recombinant expression of BDDmFc+hinge results in production of a Factor VIII fusion heterodimer (denoted as “BDDFc+hinge”) as shown in FIG. 2. Due to the presence of a functional immunoglobulin hinge region, two molecules of BDDFc+hinge covalently associate via disulfide bonding to form a multimeric Factor VIII fusion heterodimer. An advantage of this format is that dimeric Fc results in high affinity binding of the fusion protein to the FcRn, resulting in prolonged circulating half-life.

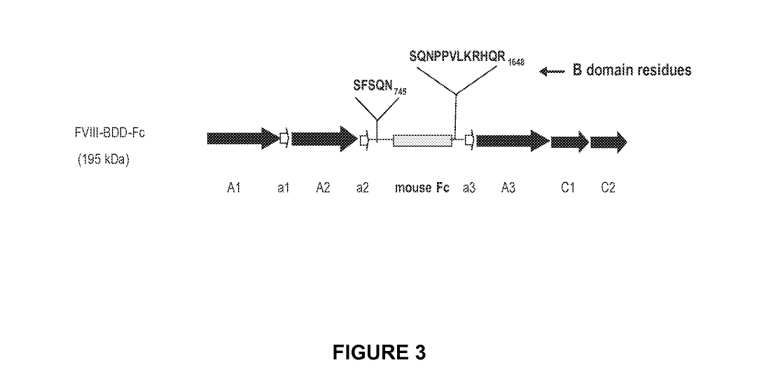

Plasmid pSK207 containing the Factor VIII B-domain deleted (BDD) gene bounded by PmeI and NheI sites (denoted “pSK207BDD”) was mutated using a site-directed mutagenesis kit. Two restriction sites (AvrII at by 4490 and AflII at by 4520) were introduced into the molecule using mutagenic primers CES16 (5′-caatgccattgaacctaggagcttctcccagaacccaccagtccttaagcgccatcaacggg-3′) (SEQ ID NO: 34) and CES17 (5′-cccgttgatggcgcttaaggactggtgggttctgggagaagctcctaggttcaatggcattg-3′) (SEQ ID NO:35). An AflII site at bp2537 was eliminated using mutagenic oligos CES18 (5′-cagtggtcattacactcaagaacatggcttccca tcc-3′) (SEQ ID NO:36) and CES19 (5′-ggatgggaagccatgttcttgag tgtaatgaccactg-3′) (SEQ ID NO:37). The resulting plasmid was designated pM109. These mutagenic events were all silent, resulting in no amino acid changes to BDD. As a source of the murine Fc region, plasmid pGT234 which contains a full-length murine IgG1 antibody against the human epidermal growth factor receptor was used. The murine Fc+hinge region was PCR amplified using primers CES 36 (5′-agcttcctaggagcttctcccagaacgtgcccagggattg tggttg-3′) (SEQ ID NO:38) and CES 39 (5′-agctacttaaggactggtgggttctgggatttaccaggagagtgggagag-3′) (SEQ ID NO:39) with pGT234 as template. The resulting fragment was digested with AflII/AvrII and cloned into AflII/AvrII-digested pM109 to produce plasmid pM117. To restore the original AflII site at by 2537, an NheI/BglII fragment of pSK207+BDD was inserted in pM117 to replace its equivalent region to produce plasmid pM115, which contains an AflII site at bp2537. The BDD.mFc+hinge gene of pM115 (contained within a 5077 bp NheI/PmeI fragment) was cloned into the PmeI/NheI sites of expression vector pSS207 to generate plasmid pM110 or pSK207BDDFc+hinge. The Factor VIII fusion gene component of pSK207BDDFc+hinge (i.e., BDDmFc+hinge) has the nucleic acid sequence of SEQ. ID NO: 31. The protein coded by BDDmFc+hinge has the structural domains illustrated in FIG. 3 (wherein “mouse Fc” indicates the location of the mFc+hinge), and the amino acid sequence of SEQ. ID NO: 32.

### Example 2

The following example describes construction of a mammalian expression vector (denoted as “pM118” or “pSS207BDDFc−hinge”) comprising a Factor VIII fusion gene (denoted as “BDDmFc−hinge”) using nucleic acid coding for a Factor VIII B-domain deleted (BDD) protein and nucleic acid coding for all but the hinge portion of a murine Fc region (denoted as “mFc−hinge”). Recombinant expression of BDDmFc−hinge results in production of a Factor VIII fusion heterodimer (denoted as “BDDFc−hinge”) as shown in FIG. 2. The protein coded by BDDmFc−hinge has the structural domains illustrated in FIG. 3 (wherein “mouse Fc” indicates the location of the mFc−hinge), and the amino acid sequence of SEQ. ID NO: 33. Due to the absence of a functional immunoglobulin hinge region, BDDFc−hinge does not form multimeric Factor VIII fusion heterodimers. A disadvantage of this format is that a non-dimerized Fc region has reduced affinity for FcRn binding.

Construction of BDDmFc−hinge is very similar to that above for BDDmFc+hinge. The mFc−hinge region was PCR amplified from plasmid pGT234 using PCR primers CES 37 (5′-agcttcctaggagcttctccca gaacgtcccagaagtatcatctgtc-3′) (SEQ ID NO:40) and CES39 (SEQ ID NO:39), digested with AvrII/AflII and cloned into the AvrII/AflII-digested pM109 plasmid. The resulting plasmid was designated pM114 (also denoted “pSK207.BDD.mFc−hinge”). The NheI/PmeI fragment of pM114 containing the BDD.mFc−hinge gene was then cloned into the expression vector pSS207 to generate the plasmid pM118 (i.e., pSS207BDDFc−hinge).

### Example 3

The following example describes construction of a plasmid (denoted “pM130”) for expression of a murine Fc region (denoted “mFc+hinge”) having a Flag tag at its amino terminal end. Coexpression of this plasmid with pSS207BDDFc+hinge in the same host cell produces a mixture of BDDFc+hinge dimers, mFc+hinge dimers, and a heterodimer of BDDFc+hinge and mFc+hinge. Inclusion of the Flag tag facilitates isolation of the heterodimer (denoted as “BDDFc”) as shown in FIG. 2 by affinity chromatography using anti-Flag antibodies and anti-Factor VIII antibodies in sequential separation steps. Those of skill in the art will appreciate; however, that even without the provision of a peptide tag, it would be possible to separate the heterodimer form using techniques well known in the art, for example, size-exclusion chromatography.

Using pM110 as template, a murine Fc region (with hinge) with a Flag tag at its 5′ (amino terminal) end was PCR-amplified using primers CES49 (5′-atatgatatcgcggccgccgccaccatggtgttgcag acccaggtcttcatttctctgttgctctggatctctggtgcctacggggactacaaagacgatgacgacaaggtgcccagggattgt ggttg-3′) (SEQ ID NO:41) and CES 50 (5′-ttcgatctcgagtcatttaccagga gagtgggagagg-3′) (SEQ ID NO:42). This fragment was digested with NotI/XhoI and ligated to the NotI/XhoI-digested expression vector pAGE16, to produce plasmid pM119 (i.e., pAGE16.mFc+hinge.Flag). Subsequently, the HindIII/XhoI fragment of pM119 containing the mFc+hinge.Flag region was subcloned into the expression plasmid pEAK fICMV W/GFP digested with HindIII/XhoI, and designated pM130.

### Example 4

The following example describes construction of Factor VIII fusion genes (denoted as “BDD.Human Fc”) using nucleic acid coding for a Factor VIII B-domain deleted (BDD) protein and nucleic acid coding for any one of the human Fc regions of IgG1, IgG2, IgG3, or IgG4, or any one of the non-hinge portion of the human Fc regions of IgG1, IgG2, IgG3, or IgG4. As an example, a Factor VIII fusion heterodimer may be generated by inserting 227 amino acid residues or 214 amino acid residues derived from mouse IgG1 Fc into a specific site (e.g., between N-745 and S-1637) of Factor FVIII to mimic the B domain.

Construction of BDD.Human Fc (from IgG1, IgG2, IgG3, or IgG4 antibodies) expression vectors follows the same strategy as that above for the BDD-murine Fc expression constructs. The pM109 plasmid is digested with AvrII/AflII and the AvrII/AflII bounded Fc+hinge and Fc−hinge is inserted into the corresponding sites. The resulting plasmids, which have a pSK backbone, are then digested with NheI and PmeI and the BDDFc fragments ligated to pSS207 for creation of stable cell clones. Similarly, a pCEP4.human Fc monomer plasmid is constructed.

Exemplary human IgG Fc region nucleic acid coding sequences include SEQ ID NO: 8 (Fc region of a human IgG1), SEQ ID NO: 10 (Fc region of a human IgG2), SEQ ID NO: 12 (Fc region of a human IgG3), and SEQ ID NO: 14 (Fc region of a human IgG4). Alternatively, a nucleic acid sequence may be used which encodes the same amino acid sequence (or an amino acid sequence having at least 90% identity) as SEQ ID NO: 9 (Fc region of a human IgG1), SEQ ID NO: 11 (Fc region of a human IgG2), SEQ ID NO: 13 (Fc region of a human IgG3), and SEQ ID NO: 15 (Fc region of a human IgG4). Exemplary non-hinge portion human IgG Fc region nucleic acid coding sequences include SEQ ID NO: 16 (non-hinge portion of the Fc region of a human IgG1), SEQ ID NO: 18 (non-hinge portion of the Fc region of a human IgG2), SEQ ID NO: 20 (non-hinge portion of the Fc region of a human IgG3), and SEQ ID NO: 22 (non-hinge portion of the Fc region of a human IgG4). Alternatively, a nucleic acid sequence may be used which encodes the same amino acid sequence (or an amino acid sequence having at least 90% identity) as SEQ ID NO: 17 (non-hinge portion of the Fc region of a human IgG1), SEQ ID NO: 19 (non-hinge portion of the Fc region of a human IgG2), SEQ ID NO: 21 (non-hinge portion of the Fc region of a human IgG3), and SEQ ID NO: 23 (non-hinge portion of the Fc region of a human IgG4).

### Example 5

The following example describes construction of a monocistronic Factor VIII fusion gene which encodes a hybrid Factor VIII fusion heterodimer. The translated Factor VIII fusion protein contains two tandem mFc+hinge regions in place of the B domain of full length FVIII.

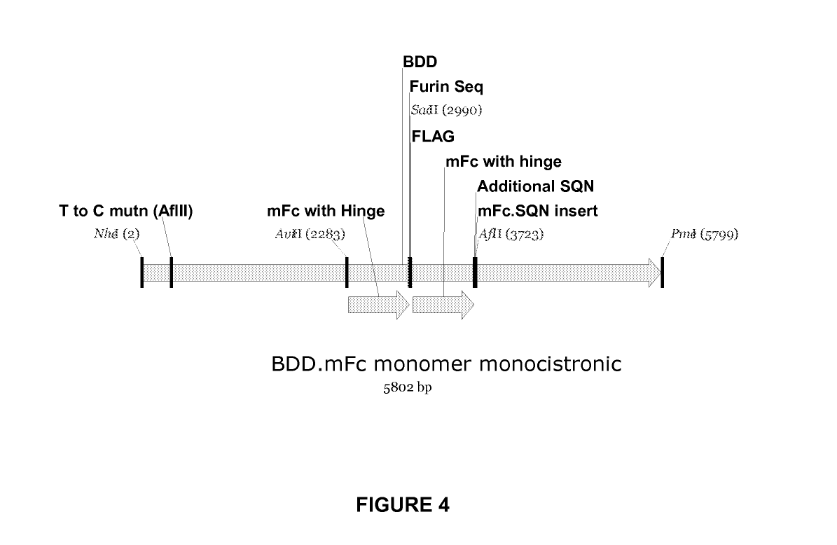

An expression plasmid is constructed as follows: Using pM117 (pSK207+BDD.mFc+hinge) as template, PCR with two sets of oligos—the first is CES36/CES51 which creates an mFc fragment bounded by AvrII (CES36: 5′-agcttcctaggagcttctcccagaacgtgcccagggattgtggttg-3′) (SEQ ID NO:30) and SacII (CES51: 5′-cagttgccgcgggctttaccaggagagtgggagagg-3′) (SEQ ID NO:35), and the second set of primers is CES52/CES39, which creates a mFc fragment bounded by SacII (CES52: 5′-ttcgcccgcggcaagagagactacaaagacgatgacgacaaggtgcccagggattgtggttg-3′) (SEQ ID NO:35) and AflII (CES39: 5′-agctacttaaggactggtgggttctgggatttaccaggagagtgggagag-3′) (SEQ ID NO:31). When appropriately digested and ligated, these two resulting PCR fragments give a monocistronic BDD gene containing, in order, A1, a1, A2, a2 domains, the first five N-terminal amino acids of the B-domain, then the mFc+hinge region, a furin consensus sequence (KARGKR (SEQ ID NO:36) with the first lysine (K) being the end of the Fc region), Flag tag (DYKDDDDK) (SEQ ID NO:37), mFc+hinge, last twelve C-terminal amino acids of the B-domain, and finally the a3, A3, C1 and C2 domains of FVIII (FIG. 4). To construct the above monomeric gene, the two PCR fragments are digested with AvrII/SacII or SacII/AflII and, via a triple ligation, cloned into pM109 (pSK207.BDD) digested with AvrII/AflII. Successful clones are sequenced and then one is cloned via NheI/PmeI from the pSK207 backbone (of pM109) to the expression vector, pSK207 digested with NheI/PmeI. During synthesis and secretion of the protein, the molecule is initially cleaved at the furin site upstream of the Flag-mFc region and at the protease site just upstream of a3. The molecule will circulate as a mature FVIII dimer (with the mFc replacing the B domain) with the Flag mFc molecule bound to the mFc region of the FVIII molecule via Fc-Fc disulphide interaction (BDDFc) as shown in FIG. 2. Heterodimeric product is isolated from any homodimeric product present in the supernatant using methods known to those skilled in the art.

### Example 6

The following example describes a general procedure useful for transient transfection of mammalian host cells and cell culturing thereof. HKB11 cells are grown in suspension culture on an orbital shaker (100-125 rpm) in a 5% CO₂ incubator at 37° C. in a protein-free medium and maintained at a density between 0.25 and 1.5×10⁶ cells/mL. HKB11 cells for transfection are collected by centrifugation at 1,000 rpm for 5 minutes, then resuspended in FreeStyle™ 293 Expression Medium (Invitrogen Corporation, Carlsbad, Calif.) at 1.1×10⁶ cells/mL. The cells are seeded in six well plates (4.6 mL/well) and incubated on an orbital rotator (125 rpm) in a 37° C. CO₂ incubator. For each well, 5 μg plasmid DNA is mixed with 0.2 ml Opti-MEM® I medium (Invitrogen Corporation, Carlsbad, Calif.). For each well, 7 μL 293Fectin™ reagent (Invitrogen Corporation, Carlsbad, Calif.) is mixed gently with 0.2 mL Opti-MEM® I medium and incubated at room temperature for 5 minutes. The diluted 293Fectin™ is added to the diluted DNA solution, mixed gently, incubated at room temperature for 20-30 minutes, then added to each well that has been seeded with 5×10⁶ (4.6 mL) HKB11 cells. The cells are then incubated on an orbital rotator (125 rpm) in a CO₂ incubator at 37° C. for 3 days after which the cells are pelleted by centrifugation at 1000 rpm for 5 minutes and the supernatant is then collected and stored at −80° C.

### Example 7

The following example describes a general procedure useful for verifying recombinant production of Factor VIII fusion heterodimer by Western blotting. Cell culture supernatant is either concentrated 10-fold by Centricon® (Millipore Corporation, Billerica, Mass.) (when no secondary antibody is used for probing) or used neat (when secondary antibody is used for probing). Fifty μL supernatant is mixed with 20 μL 4×SDS-PAGE loading dye with DTT (reducing) or without DTT (non-reducing), heated at 95° C. for 5 minutes, then loaded onto 10% NuPAGE® gels (Invitrogen Corporation, Carlsbad, Calif.) (under reducing condition) or onto 4-20% NuPAGE® gels (Bis-Tris-MOPs) (under non-reducing condition). Proteins are transferred to nitrocellulose membranes. After blocking with 5% milk/PBS for 60 minutes, the membranes are incubated with a horseradish peroxidase (HRP)-labeled rabbit polyclonal antibody against mouse IgG (H+L) or HRP-conjugated anti-Factor VIII C domain antibody. Also, the anti-human Factor VIII rabbit monoclonal antibody (Epitomics, Calif.) may be used to detect the light chain of Factor VIII. The membranes are then incubated with anti-rabbit IgG-HRP secondary antibody for 60 minutes at room temperature. After washing the blots with PBS/0.1% Tween®-20 (polyoxyethylenesorbitan monolaurate), the signal from HRP is detected using a chemiluminescent substrate (ECL) (Pierce, Rockford, Ill.) and exposure to x-ray film.

### Example 8

The following example describes a general procedure useful for measuring the concentration of Factor VIII antigen in cell culture supernatants by ELISA. Cell culture supernatants are diluted in PBS/BSA/Tween®-20 buffer to achieve a signal within the range of a standard curve. For example, Factor VIII BDD protein purified (specific activity 9,700 IU/mg) diluted in PBS/BSA/Tween®-20 may be used to create a standard curve from 100 ng/mL to 0.2 ng/mL. Diluted samples and the standards are added to an ELISA plate that is pre-coated with a polyclonal anti-Factor VIII capture antibody C2. After adding a biotinylated C2 as detection antibody, the plate is incubated at room temperature for 1 hour, washed extensively, and then developed using TMB substrate (3,3′,5,5′-tetramethylbenzidine) as described by the kit manufacturer (Pierce, Rockford, Ill.). Signal may be measured at 450 nM using a SpectraMax® plate reader (SpectraMax® 340 pc, Molecular Devices, Sunnyvale, Calif.). A standard curve is fitted to a four-parameter model, and the values of unknowns extrapolated from the curve.

As an alternative of the above procedure, which is not specific to intact Factor VIII fusion heterodimers, an ELISA assay which utilizes an anti-Factor VIII antibody as the capture antibody (or detection antibody) and an antibody specific to the half-life modulator as the detection antibody (or capture antibody).

### Example 9

The following example describes a general procedure useful for measuring the activity of Factor VIII fusion heterodimer in cell culture supernatants and purified fractions using a commercial chromogenic assay kit (Coatest® SP4 FVIII, Chromogenix, Lexington, Mass.) in a 96-well format. Triplicate samples are diluted to 25 μL in the kit assay buffer (50 mM Tris, pH 7.3, 10 mg/L ciprofloxin and 1.0% BSA) and added to wells. Then, 50 μL phospholipid, Factor IXa, Factor X solution is added to each well and incubated for 4 minutes at 37° C. on a horizontal shaker. Twenty-five μL CaCl₂ solution (25 mM) is immediately added to the wells and incubated in the same manner for 10 minutes. Chromogenic substrate solution (50 μL/well) is added and plates are incubated as before for 10 minutes before the color development is stopped by the addition of 25 μL 20% acetic acid. Individual wells are measured on a 96-well plate reader (SpectraMax® 340 pc, Molecular Devices, Sunnyvale, Calif.) at an absorbance at 405 nm. Factor VIII activity is quantitated against a purified Factor VIII B-domain deleted (BDD) standard ranging from 500-0.5 mIU/mL diluted in the same buffer as the unknowns and fit to a four-parameter model. Specific activities (IU/mg of FVIII) are calculated from the results of a Coatest® and Factor VIII ELISA.

### Example 10

The following example describes a general procedure useful for measuring the coagulation activity of Factor VIII fusion heterodimer in cell culture supernatants and purified fractions using an aPTT assay. Factor VIII coagulation activity may be determined using a aPTT assay in Factor VIII-deficient human plasma by an Electra™ 1800C automatic coagulation analyzer (Beckman Coulter Inc., Fullerton, Calif.). Briefly, three dilutions of supernatant samples in coagulation diluent are created by the instrument and 100 μL is then mixed with 100 μL FactorVIII-deficient plasma and 100 μL automated aPTT reagent (rabbit brain phospholipid and micronized silica, bioMérieux, Inc., Durham, N.C.). After the addition of 100 μL 25 mM CaCl₂ solution, the time to clot formation is recorded. A standard curve is generated for each run using serial dilutions of the same purified Factor VIII BDD used as the standard in the ELISA assay. The standard curve was linear with a correlation coefficient of 0.95 or better, and is used to determine the Factor VIII activity of the unknown samples.

### Example 11

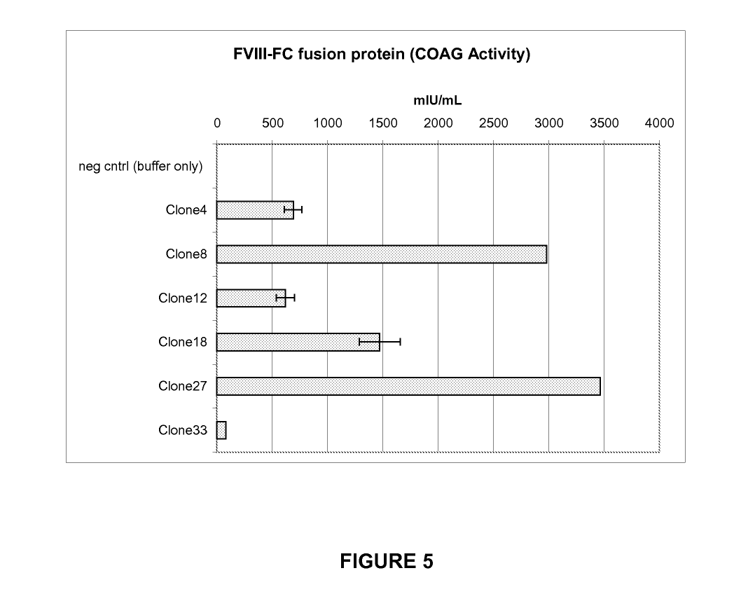

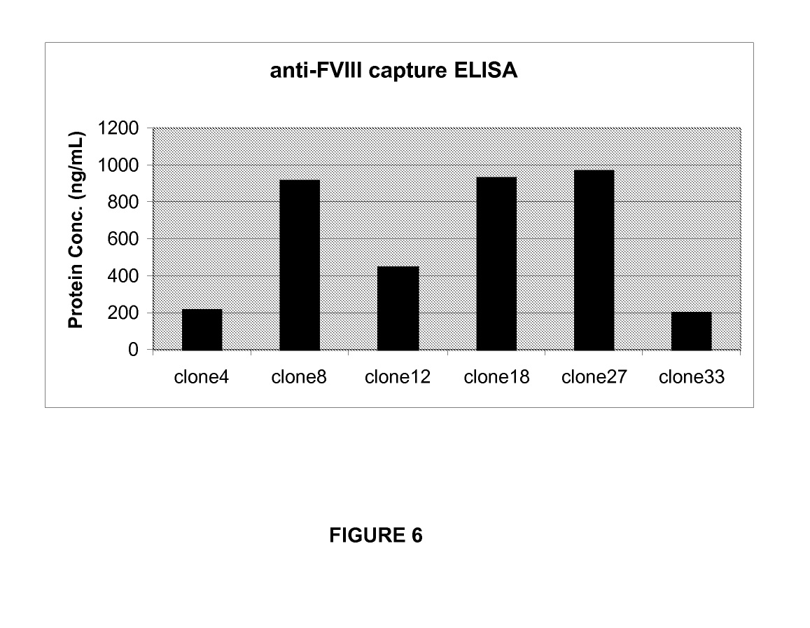

The following example describes stable transfection and creation of cell lines using the vectors described in Examples 1 and 2. HKB11 cells were transfected with plasmid DNAs, pSK207BDDFc+hinge, or pSK207BDDFc−hinge using 293Fectin™ reagent as described in Example 6. The transfected cells were split into 100-mm culture dishes at various dilutions (1:100; 1:1000; 1;10,000) and maintained in DMEM-F12 medium supplemented with 5% FBS and 200 μg/mL hygromicin (Invitrogen Corporation, Carlsbad, Calif.) for about 2 weeks. Individual single colonies were picked and transferred into 6-well plates using sterile cloning disks (Scienceware®, Bel-Art Products, Pequannock, N.J.). Over fifty clones of HKB11 cells transfected with pSK207BDDFc+hinge were established and banked. These clones were screened for high expression of Factor VIII fusion heterodimer by Factor VIII activity assays (Coatest® and aPTT assays described above) as shown in FIG. 5, by Factor VIII ELISA (described above) as shown in FIG. 6, and by growth assays. The six cell lines with highest expression levels are shown in FIG. 6. The top clone for BDDFc+hinge, Clone 8, expresses ˜1 μg/mL fusion protein when grown adherently. The specific activity of BDDFc+hinge from Clone 8 conditioned media was about 5,000-8,000 IU/mg, which is comparable to the BDD Factor VIII protein from which it is derived. Using a similar stable transfection and selection procedure, the clone (Clone t) for BDDFc−hinge was determined to express ˜1 μg/mL fusion protein when grown adherently.

### Example 12

The following example describes scale-up of protein expression by stable transformants using a 10 L WAVE Bioreactor™ (GE Healthcare, Piscataway, N.J.). Clone 8 and Clone t cells were maintained in DMEM-F12 medium supplemented with 5% FBS and 200 μg/mL hygromicin. The cells were split 1:4 every 3 days from T75 to T225 flasks. For culture adaptation, about 1,000 million cells from twelve T225 flasks were transferred into 1 L suspension media that was serum-free supplemented with 2.5% FBS in 2 L- or 3 L-Erlenmyer flasks. Two days later, cells were expanded into serum-free suspension media supplemented with 1.25% FBS. The cells were then transferred into serum-free suspension media supplemented with 5% human plasma protein solution (HPPS). Approximately 10,000-15,000 million cells were seeded at a density of about 1 million/ml in medium in a 10 L WAVE Bioreactor™ bag. Three days later, cell density had reached 5-6 million/mL, and conditioned medium was harvested. The crude medium was first clarified to remove cell debris by continuous centrifugation with a Contifuge® Stratos (Thermo Fisher Scientific, Waltham, Mass.) at 6,000 rpm and at a flow rate of 150 mL/min as controlled by a peristaltic pump. The clarified medium was mixed with Triton® X-100 (polyethylene glycol tert-octylphenyl ether) (up to 0.05%) and concentrated about 10-fold by ultrafiltration on a 10 kDa Pellicon tangential flow membrane (Millipore, Billerica, Mass.). Sucrose was added to the concentrate to 1% prior to freezing at −80° C. The specific activities of the recombinantly produced Factor VIII fusion heterodimers before purification were determined to be 10,629 IU/mg for BDDFc+hinge produced by Clone 8 and 11,122 IU/mg for BDDFc−hinge produced by Clone t.

### Example 13

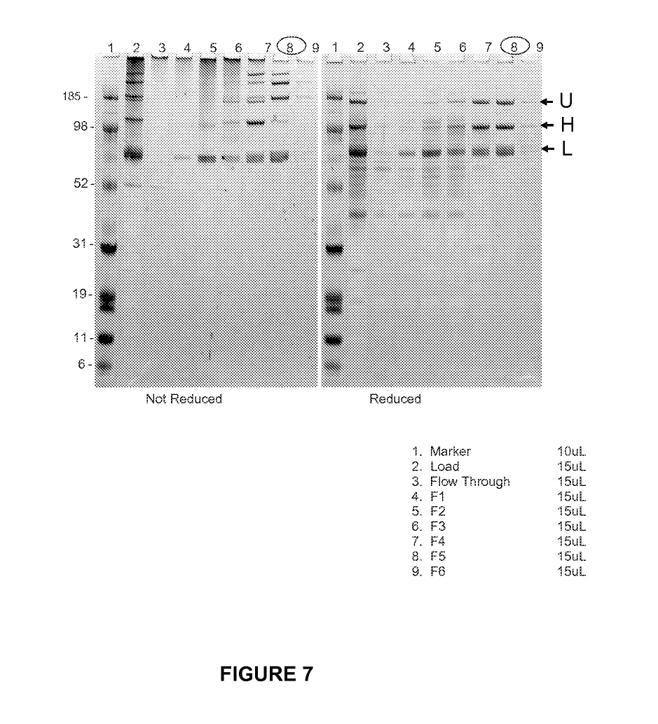

The following example describes purification of Factor VIII fusion heterodimer from the scale-up culture of Clone 8. Factor VIII BDDFc+hinge was purified from HKB11 cell conditioned media using an anti-Factor VIII monoclonal antibody affinity column (C7F7) followed by an anion exchange Q-Sepharose™ column (GE Healthcare, Piscataway, N.J.). The total recovery approached 30%. Frozen concentrate from 10 L WAVE Bioreactor™ bags was thawed and loaded onto the immunoaffinity column at 1 mL/min using an ÄKTA™ Purifier system (Amersham Pharmacia, Uppsala, SW) and then the column was washed with buffer (20 mM imidazole, 0.01 M CaCl₂, 0.5 M NaCl, 0.01% Tween®-80 (polyethylene glycol sorbitan monooleate), pH 7.0). Bound Factor VIII BDDFc+hinge was eluted with buffer containing 1.0 M CaCl₂. Fractions were assayed for Factor VIII activity by Coatest® assay and active fractions were pooled and buffer exchanged on a HiTrap™ 26/10 desalting column G25M (Amersham Biosciences, Uppsala, SW) into an ion exchange loading buffer (20 mM imidazole, 10 mM CaCl₂, 200 mM NaCl, 0.01% Tween®-80, pH 7.0). Protein was loaded onto a 1 ml HiTrap™ Q HP column (Amersham Biosciences, Uppsala, SW), and eluted with a NaCl gradient (200 mM-1000 mM). Fractions were assayed for Factor VIII activity by Coatest® assay and peak fractions pooled. Protein concentration and specific activity were determined. The purity of the best fraction (i.e., Fraction 5 in lane 8 of FIG. 7) is about 80% as estimated by SDS-PAGE and SimplyBlue™ staining (Invitrogen, Carlsbad, Calif.). The purified fusion proteins contained an Fc domain since they were detected by the anti-Fc antibody in Western blot analyses. The specific activity of the purified material was about 10,000 IU/mg. This specific activity is very comparable to Factor VIII BDD (from which BDDFc+hinge is derived) suggesting that BDDFc+hinge is fully active.

### Example 14

The following example describes an endotoxin test on a recombinantly produced Factor VIII fusion heterodimer. Endotoxin levels of purified protein solutions were determined using a kinetic chromogenic Limulus Amebocyte Lysate assay (Endosafe® kit) with a sensitivity of 0.005 EU/mL. The levels of endotoxin in BDDFc+hinge were found to be 1.3-2.0 EU/mL which is well below 5 EU/dose.

### Example 15

The following example describes pharmacokinetic studies in normal mice using purified BDDFc+hinge, purified BDDFc−hinge, and the Factor VIII protein (“BDD-FVIII”) from which these Factor VIII fusion heterodimers are derived. Normal C57 male mice were intravenously injected with a single dose of BDD and fusion proteins (BDDFc+hinge or BDDFc−hinge) at 50 μg/kg body weight. Blood samples were collected at t=0, 0.083, 0.5, 2, 4, 6, 8, 24, 28, 32, 48, and 72 hours post injection (5 mice per time point). Both protein levels (by antigen ELISA) and coagulation activity (by Coatest® assay) in the blood samples were determined for pharmacokinetic analyses. The results are reported in Table 1.

TABLE 1

| | T_{1/2} | Clₜₒₜₐₗ | Vₛₛ | |
|---|---|---|---|---|
| | (hour) | (mLh/kg) | (mL/kg) | AUC/D |
| BDD-FVIII | | | | |
| ELISA | 5.4 | 11.2 | 75 | 89 |
| Coatest ® Activity | 3.7 | 13.7 | 60 | |
| BDDFc + hinge | | | | |
| ELISA | 3.2 | 49 | 176 | 20.4 |
| Coatest ® Activity | 2.5 | 73 | 136 | |
| BDDFc − hinge | | | | |
| ELISA | 4.7 | 23 | 128 | 43.7 |
| Coatest ® Activity | 4.3 | 29 | 136 | |

The beta half-life of BDDFc+hinge and BDDFc−hinge was similar to BDD-FVIII in normal mice.

### Example 16

The following example describes pharmacokinetic studies in a hemophilia A animal model (HemA mice) using purified BDDFc−hinge, and the Factor VIII protein (“BDD-FVIII”) from which BDDFc−hinge is derived. The results indicate that BDDFc−hinge has a significantly prolonged beta phase half-life in Hem A mice in comparison with BDD-FVIII.

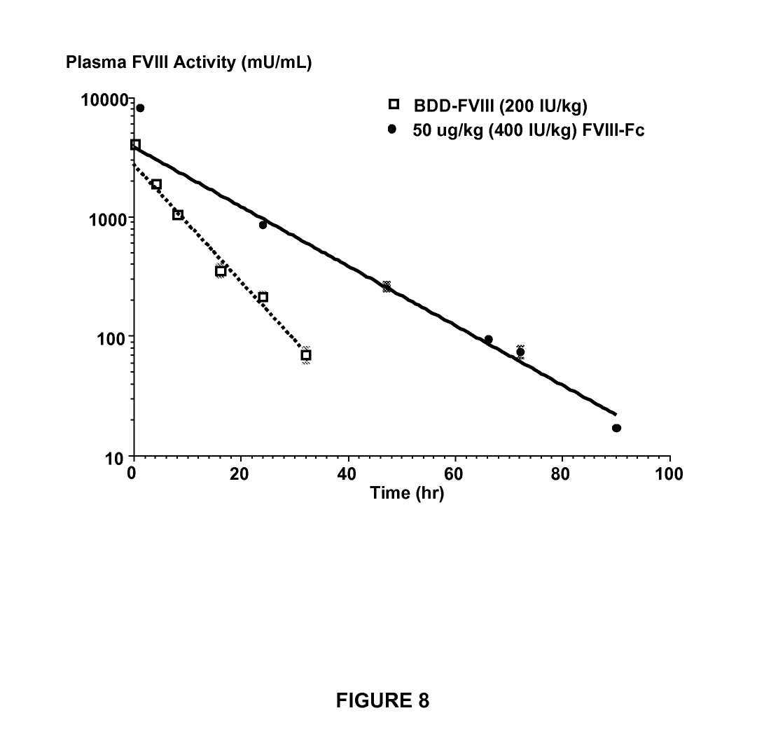

HemA mice were injected via the tail vein (i.v.) with BDDFc−hinge (“FVIII-Fc,” 9 mice) at 1.25 μg/mouse (50 μg/kg) in formulation buffer containing 5% albumin. Additional HemA mice received 200 IU/kg BDD-FVIII; the Factor VIII variant from which BDDFc−hinge is derived. Blood was collected in citrate via the retro-orbital at 1, 24, 48, 66, 72, 90, 120, and 148 hrs from alternating mice (3 mice/time point) that received BDDFc−hinge, and at 1, 4, 8, 16, 24, and 32 hrs from alternating mice (5 mice/time point) that received BDD-FVIII. Plasma FVIII activity was measured using Coatest® SP FVIII kit (Instrumentation Laboratory Company, Lexington, Mass.). Beta phase half-life was estimated by sparse-sampling and the non-compartment model in WinNonlin® (Pharsight, Mountain View, Calif.). For Coatest® assays, BDD-FVIII was used to generate the standard curve. Briefly, samples, standards, positive, and negative controls (25 μL each) in the same plasma matrix were added in duplicates to a 96-well plate. A mixture (50 μL) of FIXa, FX, and phospholipid solution was added and incubated at 37° C. for 5 minutes. Then, 25 μL CaCl₂ solution was added and incubated at 37° C. for 5 minutes, followed by addition of 50 μL substrate and incubation at 37° C. for approximately 5 minutes until color developed to proper intensity. Stop solution (25 μL) was added and the plate was read at OD 405 nm on a plate reader (SpectraMax® 250, Molecular Devices, Sunnyvale, Calif.). The results were calculated using SoftMax® Pro 4.8 (Molecular Devices, Sunnyvale, Calif.) as shown in FIG. 8. Results presented are mean±SD from 5 mice for BDD-FVIII, and from 3 mice for FVIII-Fc, at each time point.

In comparison to the decay curve of BDD-FVIII, BDDFc−hinge showed biphasic decay with a rapid distribution phase (FIG. 8). The beta phase half-life of BDDFc−hinge was 11.9 hrs at 50 μg/kg, which is about a two-fold improvement relative to unmodified BDD-FVIII with a beta phase half-life is 6.03 hrs. There may be a possibility that some Factor VIII fusion heterodimers may not be analyzed using pharmacokinetic studies in a non-hemophilia A animal model.

### Example 17

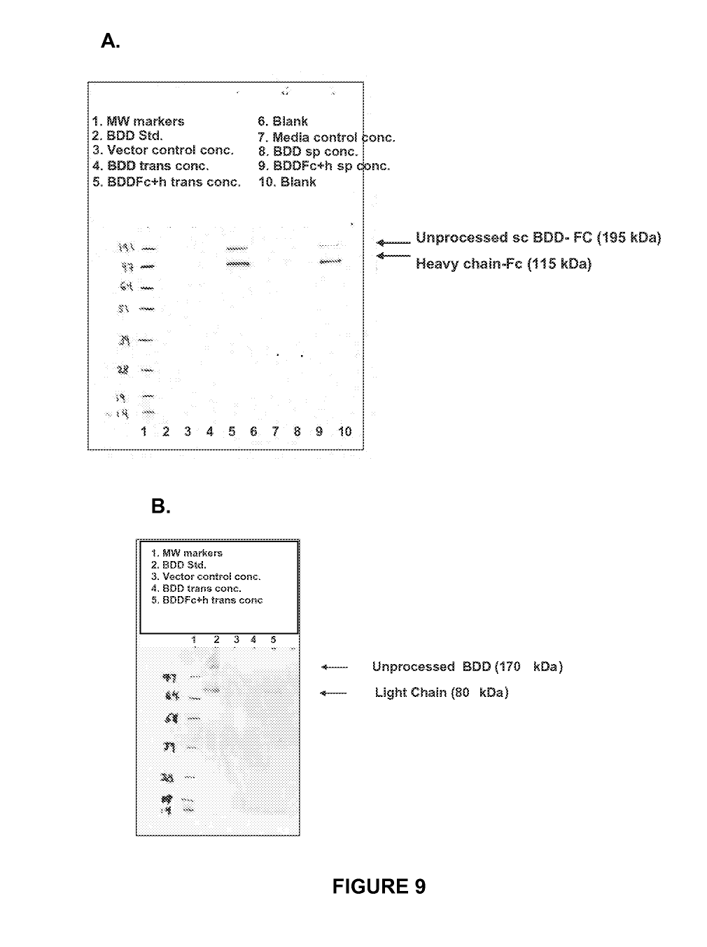

The following example describes in vitro studies on recombinant Factor VIII fusion heterodimers which are the expression product of the Factor VIII fusion genes described above. The mammalian expression vectors pSS207BDDFc+hinge and pSS207BDDFc−hinge were transiently transfected into HKB11 cells and conditioned medium was collected 72 hours post-transfection as described above. As shown in FIG. 9A, Western blot analysis of concentrated supernatants under reducing conditions showed that BDDFc+hinge Factor VIII fusion heterodimers were initially expressed as an ˜195 kDa Factor VIII fusion protein as detected by anti-Fc antibody (lane 5) which was post-translationally processed into a 115-kDa heavy chain as detected by anti-Fc antibody (lane 5); and as shown in FIG. 9B, a 80-kDa light chain as detected by Factor VIII light chain specific antibody (lane 5). For comparison, purified BDD protein and conditioned media from HKB11 cells transiently transfected with pSK207 or pSK207BDD (an expression vector comprising the B-domain deleted Factor VIII gene from which the Factor VIII fusion gene coding for BDDFc+hinge was derived) did not react with anti-Fc antibody (FIG. 9A), and using a Factor VIII light chain antibody, purified BDD protein or conditioned media from HKB11 cells transiently transfected with pSK207BDD identified an expected 80 kDa light chain (FIG. 9B). In contrast, no light chain was detected in conditioned media from HKB11 cells transiently transfected with pSK207 (FIG. 9B). These results indicate that insertion of an Fc region into a deleted B domain region did not affect post-translational modification, as the molecular weight of the light chain would not be expected to change since the Factor VIII fusion gene encoding BDDFc+hinge still retained a functional cleavage site at the B-a3 domain junction.

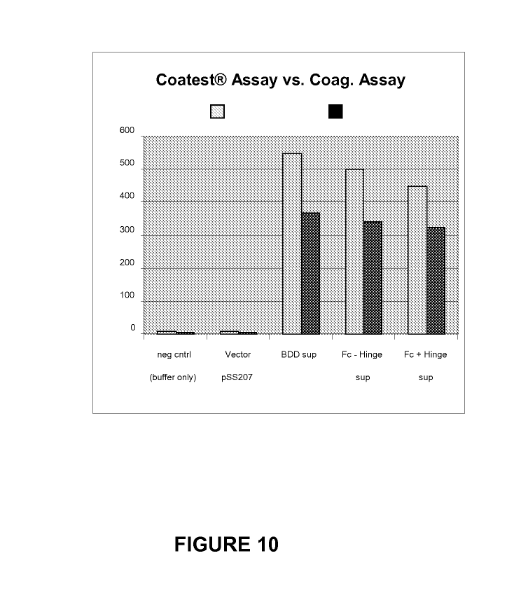

Factor VIII activity was detected in the conditioned medium from pSK207BDD (control), pSK207BDDFc+hinge, and pSK207BDDFc−hinge transfectants by Coatest® assays and by aPPT coagulation assays (FIG. 10). No Factor VIII activity was detected in conditioned media from pSK207 transfectants. The activity range of both BDDFc fusion proteins (i.e., BDDFc+hinge and BDDFc−hinge) was comparable to BDD. The data suggested that insertion of an Fc region into the specific site used did not affect the post-translational processing or biological activity of the Factor VIII fusion heterodimers in comparison to the BDD Factor VIII protein from which they were derived.

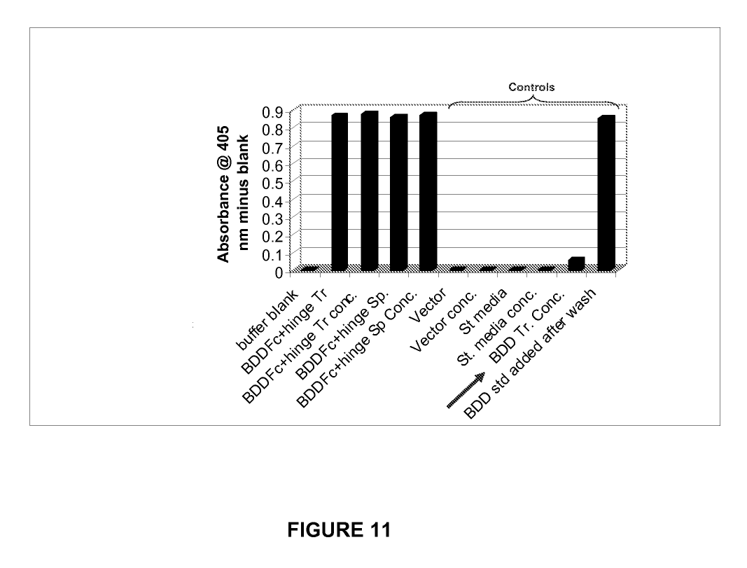

A solid phase Coatest® assay in which conditioned medium collected from HKB11 cells transiently transfected with pSK207BDDFc+hinge or pSK207BDD, was added to a 96-well plate pre-coated with rabbit-anti-mouse Fc antibody (Pierce, Rockford, Ill.), to capture the Factor VIII fusion heterodimers. Only the BDDFc+hinge fusion protein would bind to the plate and Factor VIII BDD protein from which it is derived is washed away. The Coatest® assay was then performed directly on the BDDFc+hinge immobilized to the wells, and FIG. 11 shows that BDDFc+hinge was active in this assay.

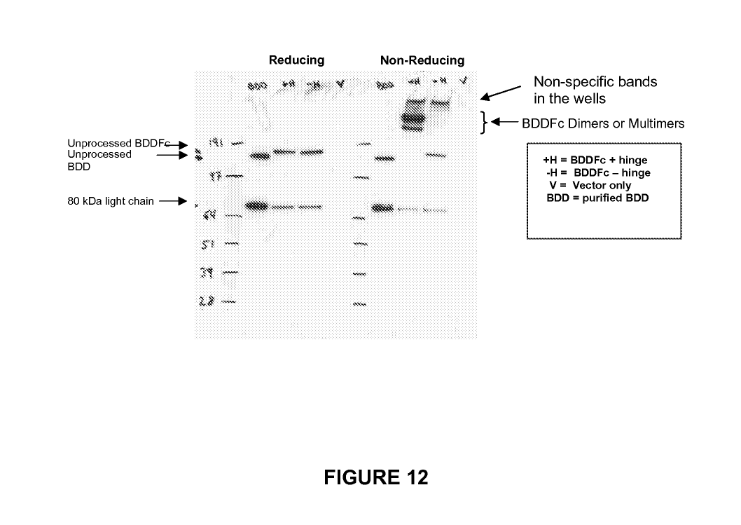

Analyses were performed using 5-fold concentrated conditioned media from HKB11 cells transiently transfected with pSK207BDDFc+hinge or pSK207BDDFc−hinge expression vectors. Samples were separated on 4-12% NuPAGE® gels under reducing and non-reducing conditions. The blot was probed with rabbit monoclonal anti-FVIII light chain antibody (Epitomics, Burlingame, Calif.) followed by HRP-conjugated anti-rabbit IgG secondary antibody. Results indicated that BDDFc+hinge forms dimers (i.e., a mutimeric Factor VIII fusion heterodimer), whereas BDDFc−hinge is a monomer (FIG. 12). Similar results were seen with cells stably transformed with pSK207BDDFc+hinge.

### Example 18

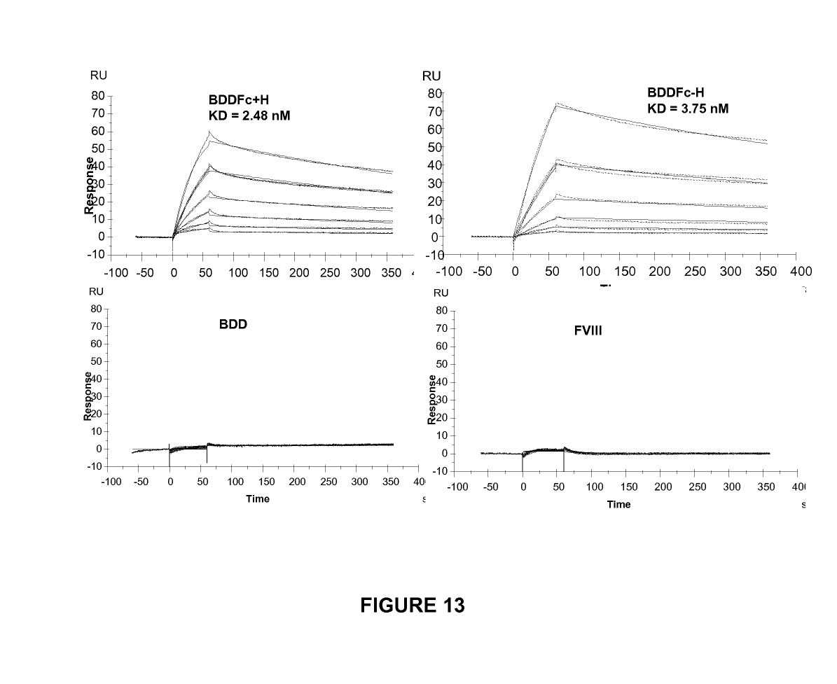

The following example describes functional studies performed using the Biacore™ system to determine whether an FcRn binding epitope retains it ability to bind to FnRn when incorporated in a Factor VIII fusion heterodimer. For use in the Biacore™ test, recombinant mouse FcRn (mFcRn) protein was expressed in CHO-K1 cells and purified by mouse IgG-affinity chromatography. Mouse FcRn was immobilized onto a CM-5 chip by amine coupling. Two Factor VIII heterodimers (BDDFc+hinge and BDDFc−hinge), BDD (the Factor VIII protein from which BDDFc+hinge and BDDFc−hinge are derived), and full-length recombinant Factor VIII were passed over the surface of the chip at various concentrations (e.g., 1.5, 3, 6, 12, 25, and 50 nM). Binding of BDDFc±hinge Factor VIII fusion heterodimers to immobilized mFcRn was detected (FIG. 13). A binding affinity (KD=2.48 nM) was calculated for BDDFc+hinge (“BDDFc+H”) and BDDFc−hinge (“BDDFc−H”) was similar to that of BDDFc+hinge (KD=3.75 nM). No detectable binding was seen with BDD (“BDD”) or full-length recombinant Factor VIII (“FVIII”).

The results indicate that BDDFc+hinge and BDDFc−hinge Factor VIII fusion heterodimers exhibit strong binding for mFcRn with nM affinity. In contrast, neither BDD nor full-length recombinant FVIII were able to bind mFcRn which is expected since they do not contain the FcRn binding epitope. In view of the pharmacokinetic studies performed using Hem A mice, the results suggest that BDDFc+hinge and BDDFc−hinge contain a functional FcRn binding epitope that binds to mFcRn with high affinity, leading to a prolonged beta phase half-life in vivo.

### Example 19

In circulation, FVIII is mainly bound to von Willebrand factor (vWF) as a stable complex. Upon activation by thrombin (Factor IIa), FVIII dissociates from the complex to interact with the coagulation cascade. Activated FVIII is proteolytically inactivated in the process (most prominently by activated Protein C and Factor IXa) and quickly cleared from the blood stream. The following example describes functional studies performed using Biacore™ system to determine whether a Factor VIII protein retains it ability to bind to von Willebrand Factor (vWF) when incorporated in a Factor VIII fusion heterodimer.

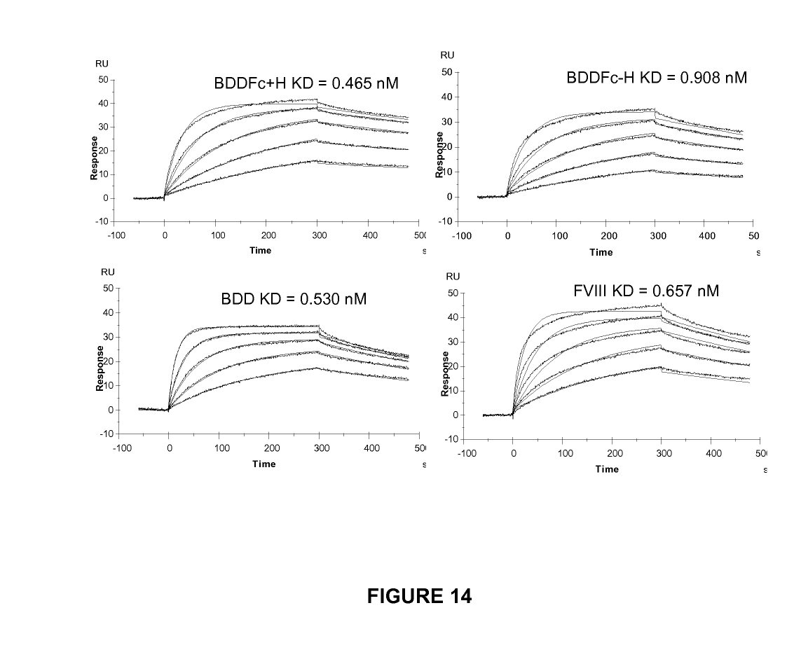

Human vWF was immobilized onto a CM-5 chip by amine coupling. Two Factor VIII heterodimers (BDDFc+hinge and BDDFc−hinge), BDD (the Factor VIII protein from which BDDFc+hinge and BDDFc−hinge are derived), and full-length recombinant Factor VIII were passed over the surface of the chip at various concentrations (e.g., 1, 2, 4, 8, 16, and 25 nM). Both BDD (“BDD”) and full-length recombinant Factor VIII (“FVIII”) were able to bind human vWF at sub-nanomolar affinity (0.53-0.657 nM) and the binding of BDDFc+hinge (“BDDFc+H”) or BDDFc−hinge (“BDDFc−H”) to vWF was also detected (FIG. 14). The binding affinity (KD) of BDDFc+hinge and BDDFc−hinge was calculated as 0.465 nM and 0.908 nM, respectively. The data shows that the Factor VIII fusion heterodimers BDDFc+hinge and BDDFc−hinge have sub-nanomolar affinity for vWF and the use of an immunoglobulin Fc region as a modulator does not block the binding properties of BDD to vWF.

### Example 20

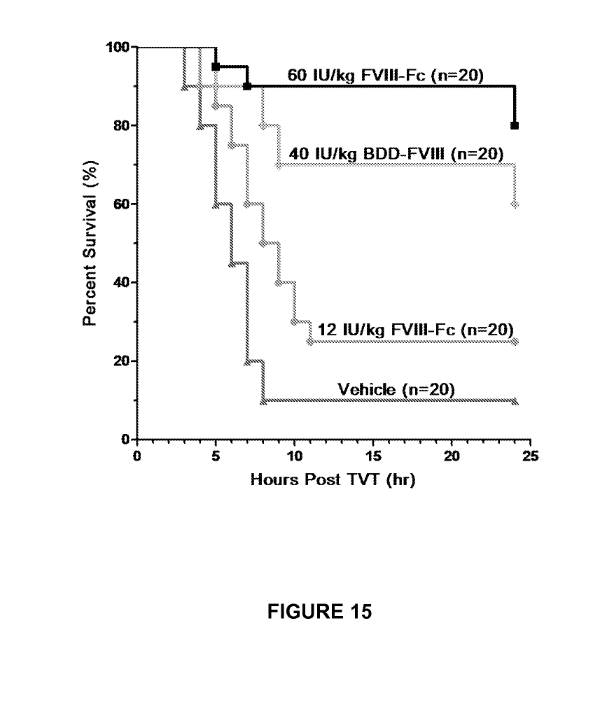

The following example demonstrates that BDDFc−hinge was efficacious in the tail vein transection bleeding model of HemA mice. HemA mice (8-10 weeks, ˜25 g) were injected via the tail vein 100 μL BDDFc−hinge in a formulation buffer containing 5% albumin at a final dose of 12 or 60 IU/kg, or 100 μL BDD-FVIII in formulation buffer containing 5% albumin at 40 IU/kg, or formulation buffer alone (vehicle) (20 mice/treatment group) at 48 hours prior to the transection of one lateral tail vein. Mice were anesthetized (with Ketamine/Xylazine), and one lateral tail vein was transected at place where the diameter of the tail was approximately 2.7 mm. The tail was then rinsed with saline pre-warmed to 37° C. until clotted, and the bleeding time was recorded. Mice were then transferred into individual cages with paper bedding on top of a heating pad, and were observed hourly for the first 9 hours and then at 24 hours post injury. Incidents of rebleeding were recorded. Statistic analysis was performed in GraphPad Prism® 4 and results are reported in FIG. 15.

In comparison to the vehicle-control group in which only 10% survived for 24 hrs following the injury, 12 IU/kg and 60 IU/kg of BDDFc−hinge achieved 25% and 80% survival, respectively. The efficacy of FVIII-Fc−hinge is estimated to be comparable to that of BDD-FVIII, which resulted in 60% survival at 40 IU/kg. All treatments resulted in significantly improved (2-tailed p<0.05 by Log-Rank test) survival curves vs vehicle control.

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A Factor VIII fusion protein or a Factor VIII fusion heterodimer comprising a Factor VIII protein or polypeptide in which an amino acid sequence of a modulator is present in the B-domain, or an amino acid sequence of a modulator replaces some or all of the amino acid sequence of the B-domain.

2. The Factor VIII fusion protein or Factor VIII fusion heterodimer of claim 1, wherein the modulator is a half-life modulator.

3. The Factor VIII fusion protein or Factor VIII fusion heterodimer of claim 1, wherein the amino acid sequence of the modulator is glycosylated.

4. The Factor VIII fusion protein or Factor VIII fusion heterodimer of claim 1, wherein the modulator is an Fc region of an immunoglobulin or variant thereof, or a FcRn binding peptide or a variant thereof.

5. The Factor VIII fusion protein or Factor VIII fusion heterodimer of claim 1, wherein the modulator is an Fc region of an immunoglobulin obtained from human IgG, IgE, IgD or IgM or a variant thereof, or mouse IgG, IgA, IgM, or a variant thereof.

6. The Factor VIII fusion protein or Factor VIII fusion heterodimer of claim 1, wherein the Factor VIII protein or polypeptide has some or all of the B-domain deleted.

7. The Factor VIII fusion protein or Factor VIII fusion heterodimer of claim 1, comprising a first amino acid sequence identical to amino acids 20-764 of SEQ ID NO: 1, a second amino acid sequence identical to amino acids 1656-2351 of SEQ ID NO:1 and a modulator amino acid sequence in which (1) the modulator amino acid sequence is covalently attached at its amino terminal to the carboxyl terminal of the first amino acid sequence and covalently attached at its carboxyl terminal to the amino terminal of the second amino acid or (2) the modulator amino acid sequence is covalently attached at its amino terminal to the carboxyl terminal of the first amino acid sequence and the modulator amino acid sequence is not covalently attached to the second amino acid sequence.

8. The Factor VIII fusion protein or Factor VIII fusion heterodimer of claim 6, wherein the modulator is an Fc region of an immunoglobulin or variant thereof, or a FcRn binding peptide or variant thereof.

9. The Factor VIII fusion protein or Factor VIII fusion heterodimer of claim 6, wherein the modulator is an Fc region of an immunoglobulin obtained from human IgG, IgE, IgD or IgM, or mouse IgG, IgA, IgM, or a variant thereof.

10. A nucleic acid encoding a Factor VIII fusion protein, wherein the Factor VIII fusion protein comprises a Factor VIII protein in which an amino acid sequence of a modulator is present in the B-domain, or an amino acid sequence of a modulator replaces some or all of the amino acid sequence of the B-domain.

11. The nucleic acid of claim 10, wherein the modulator is an Fc region of an immunoglobulin or variant thereof, or a FcRn binding peptide or a variant thereof.

12. The nucleic acid of claim 10, wherein the modulator is an Fc region of an immunoglobulin obtained from human IgG, IgE, IgD or IgM, or mouse IgG, IgA, IgM, or a variant thereof.

13. The nucleic acid of claim 10, wherein the Factor VIII protein has some or all of the B-domain deleted.

14. The nucleic acid of claim 10, wherein the Factor VIII fusion protein comprises a first amino acid sequence identical to amino acids 20-764 of SEQ ID NO: 1, a second amino acid sequence identical to amino acids 1656-2351 of SEQ ID NO:1 and a modulator amino acid sequence in which the modulator amino acid sequence is covalently attached at its amino terminal to the carboxyl terminal of the first amino acid sequence and covalently attached at its carboxyl terminal to the amino terminal of the second amino acid.

15. The nucleic acid of claim 14, wherein the modulator is an Fc region of an immunoglobulin or variant thereof, or a FcRn binding peptide or a variant thereof.

16. The nucleic acid of claim 15, wherein the modulator is an Fc region of an immunoglobulin obtained from human IgG, IgE, IgD or IgM, or mouse IgG, IgA, IgM, or a variant thereof.

17. A vector comprising the nucleic acid of claim 10.

18. A host cell comprising the nucleic acid of claim 10.

19. A method for producing the Factor VIII fusion protein or Factor VIII fusion heterodimer of claim 1, comprising (a) providing a host cell transformed with an expression vector encoding the Factor VIII fusion protein or Factor VIII fusion heterodimer; (b) culturing the cell; and (c) isolating the Factor VIII fusion protein or Factor VIII fusion heterodimer.

20. The method of claim 19, wherein the host cell is a mammalian host cell and the amino acid sequence of the modulator is glycosylated.

21. The method of claim 19, wherein the modulator is an Fc region of an immunoglobulin or variant thereof, or a FcRn binding peptide or a variant thereof.

22. The method of claim 19, wherein the Factor VIII fusion protein or Factor VIII fusion heterodimer comprises first amino acid sequence identical to amino acids 20-764 of SEQ ID NO: 1, a second amino acid sequence identical to amino acids 1656-2351 of SEQ ID NO:1 and a modulator amino acid sequence in which (1) the modulator amino acid sequence is covalently attached at its amino terminal to the carboxyl terminal of the first amino acid sequence and covalently attached at its carboxyl terminal to the amino terminal of the second amino acid or (2) the modulator amino acid sequence is covalently attached at its amino terminal to the carboxyl terminal of the first amino acid sequence and the modulator amino acid sequence is not covalently attached to the second amino acid sequence.

23. A pharmaceutical composition comprising the Factor VIII fusion protein or Factor VIII fusion heterodimer of claim 1 and a pharmaceutically acceptable carrier.

24. A method of treating genetic and acquired deficiencies in coagulation comprising administering a therapeutically effective amount of the pharmaceutical composition of claim 23 to a patient in need thereof.

25. The method of claim 24, wherein the genetic and acquired deficiencies in coagulation are hemophilia A.

26. The Factor VIII fusion protein or Factor VIII fusion heterodimer of claim 5, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

27. The Factor VIII fusion protein or Factor VIII fusion heterodimer of claim 26, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30.

28. The Factor VIII fusion protein or Factor VIII fusion heterodimer of claim 9, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

29. The Factor VIII fusion protein or Factor VIII fusion heterodimer of claim 28, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30.

30. The nucleic acid of claim 12, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

31. The nucleic acid of claim 30, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30.

32. The nucleic acid of claim 16, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

33. The nucleic acid of claim 32, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30.

34. A vector comprising the nucleic acid of claim 31.

35. A host cell comprising the nucleic acid of claim 31.

36. A vector comprising the nucleic acid of claim 33.

37. A host cell comprising the nucleic acid of claim 33.

38. The method of claim 21, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

39. The method of claim 38, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30.

40. The method of claim 21, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

41. The method of claim 22, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

42. The method of claim 40, wherein the modulator has an amino acid sequence selected from the group consisting of SEQ ID NOS: 9, 11, 13, 15, 29, 17, 19, 21, 23, 30, and sequences having at least 95% amino acid identity with any one of SEQ ID NOS: 9, 11, 13, 15, 29, 17, 19, 21, 23, 30. The method of claim 20, wherein the modulator is an Fc region of a human or mouse IgG, a variant of an Fc region of a human or mouse IgG which has a non-functional hinge (by substitution or deletion of cysteine(s) residues in the hinge region), or the non-hinge portion of an Fc region of a human or mouse IgG.

43. The Factor VIII fusion protein or Factor VIII fusion heterodimer of any of claims 3-8, wherein the modulator is a half-life modulator.

44. The nucleic acid of claim 10, wherein the modulator is a half-life modulator.

45. The method of claim 19, wherein the modulator is a half-life modulator.
